SEQUENCE LISTING (30 sequences)

SEQ ID NO: 1 - protein, 118 aa, Mus musculus [sequence omitted]

SEQ ID NO: 2 - protein, 107 aa, Mus musculus
DVVMTQSHKFMSTSVGDRVSITCKASQDVSTAVAWYQQKSGQSPKLLIHSASYRYTGVPDRFTGSGSGTDFTFTISSVQAEDLAVYYCQQHYSIPLTFGAGTKLELK

SEQ ID NO: 3 - protein, 12 aa, Mus musculus
VSGFSLTGYGVN

SEQ ID NO: 4 - protein, 12 aa, Mus musculus
WLGMIWGDGRTD

SEQ ID NO: 5 - protein, 12 aa, Mus musculus
ASDYYGSGSFAY

SEQ ID NO: 6 - protein, 11 aa, Mus musculus
KASQDVSTAVA

SEQ ID NO: 7 - protein, 10 aa, Mus musculus
LLIHSASYRY

SEQ ID NO: 8 - protein, 9 aa, Mus musculus
QQHYSIPLT

SEQ ID NO: 9 - protein, 118 aa, synthetic construct
QITLKESGPTLVKPTQTLTLTCTVSGFSLTGYGVNWIRQPPGKALEWLGMIWGDGRTDYSPSLKSRLTITKDTSKNQVVLTMTNMDPVDTATYYCASDYYGSGSFAYWGQGTTVTVSS

SEQ ID NO: 10 - protein, 118 aa, synthetic construct
QITLKESGPTLVKPTQTLTLTCTVSGFSLTGYGVNWIRQPPGKALEWLGMIWGDGRTDYSPSLKSRLTITKDTSKNQVVLTMTNMDPVDTATYYCASDYYGSGSFAYWGRGTLVTVSS

SEQ ID NO: 11 - protein, 118 aa, synthetic construct
EVQLVESGGGLVQPGGSLRLSCSVSGFSLTGYGVNWVRQAPGKGLEWLGMIWGDGRTDYADSVKGRFTISRDNSKNTLYLQMSSLRAEDTAVYYCASDYYGSGSFAYWGQGTLVTVSS

SEQ ID NO: 12 - protein, 107 aa, synthetic construct
DIQMTQSPSSLSASVGDRVTITCKASQDVSTAVAWYQQKPGKAPKLLIHSASYRYTGVPSRFSGSGSGTDFTFTISSLQPEDIATYYCQQHYSIPLTFGQGTKVEIK

SEQ ID NO: 13 - protein, 107 aa, synthetic construct
DIVMTQTPLSLSVTPGQPASISCKASQDVSTAVAWYLQKPGQPPQLLIHSASYRYTGVPDRFSGSGSGTDFTLKISRVEAEDVGVYYCQQHYSIPLTFGPGTKVDIK

SEQ ID NO: 14 - protein, 107 aa, synthetic construct
EIVMTQSPATLSLSPGERATLSCKASQDVSTAVAWYQQKPGQAPRLLIHSASYRYTGIPARFSGSGSGTDFTLTISSLQPEDFAVYYCQQHYSIPLTFGQGTKVEIK

SEQ ID NO: 15 - protein, 107 aa, synthetic construct
DIVMTQSPDSLAVSLGERATINCKASQDVSTAVAWYQQKPGQPPKLLIHSASYRYTGVPDRFSGSGSGTDFTLTISSLQAEDVAVYYCQQHYSIPLTFGQGTRLEIK

SEQ ID NO: 16 - protein, 107 aa, synthetic construct
AIQLTQSPSSLSASVGDRVTITCKASQDVSTAVAWYQQKPGKAPKLLIHSASYRYTGVPSRFSGSGSGTDFTLTISSLQPEDFATYYCQQHYSIPLTFGQGTKVEIK

SEQ ID NO: 17 - protein, 467 aa, synthetic construct
  SIGNAL 1..18
MGWSCIILFLVATATGVHCQITLKESGPTLVKPTQTLTLTCTVSGFSLTGYGVNWIRQPPGKALEWLGMIWGDGRTDYSPSLKSRLTITKDTSKNQVVLTMTNMDPVDTATYYCASDYYGSGSFAYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 18 - protein, 467 aa, synthetic construct
  SIGNAL 1..18
MGWSCIILFLVATATGVHCQVQLVESGGGLVQPGGSLRLSCSVSGFSLTGYGVNWVRQAPGKGLEWLGMIWGDGRTDYADSVKGRFTISRDNSKNTLYLQMSSLRAEDTAVYYCASDYYGSGSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 19 - protein, 467 aa, synthetic construct
  SIGNAL 1..18: note = signal peptide
MGWSCIILFLVATATGVHCQVQLVESGGGLVQPGRSLRLSCTVSGFSLTGYGVNWFRQAPGKGLEWLGMIWGDGRTDYTASVKGRFTISRDGSKSIAYLQMNSLKTEDTAVYYCASDYYGSGSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 20 - protein, 233 aa, synthetic construct
  SIGNAL 1..18: note = signal peptide
MGWSCIILFLVATATGVHSDIVMTQTPLSLSVTPGQPASISCKASQDVSTAVAWYLQKPGQPPQLLIHSASYRYTGVPDRFSGSGSGTDFTLKISRVEAEDVGVYYCQQHYSIPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 233 aa, synthetic construct
  SIGNAL 1..18
MGWSCIILFLVATATGVHSDIVMTQSPDSLAVSLGERATINCKASQDVSTAVAWYQQKPGQPPKLLIHSASYRYTGVPDRFSGSGSGTDFTLTISSLQAEDVAVYYCQQHYSIPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 233 aa, synthetic construct
  SIGNAL 1..18
MGWSCIILFLVATATGVHSEIVMTQSPATLSLSPGERATLSCKASQDVSTAVAWYQQKPGQAPRLLIHSASYRYTGIPARFSGSGSGTDFTLTISSLQPEDFAVYYCQQHYSIPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 1404 bp, synthetic construct
  source 1..1404: mol_type = other DNA
  sig_peptide 1..54
atgggatggtcatgtatcatcctttttctagtagcaactgcaaccggtgtacattgccaaatcacactgaaggagagcggaccaaccctggtgaagccaacccagacactgaccctgacatgcaccgtctctggctttagcctgacaggctacggcgtgaactggatcagacagccacctggcaaggccctggagtggctgggaatgatctggggcgacggacggacagattatagccccagcctgaaatccagactgaccatcaccaaggacacctctaagaaccaggtggtgctgacaatgaccaatatggaccccgtggatacagccacctactattgcgcctcagactattacggctcaggttcgttcgcatactggggccagggtaccacggtcacggtgtcttctgcttcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgctgcatgaggctctgcacagccactacacgcagaagagcctctccctgtccccgggtaaatga

SEQ ID NO: 24 - DNA, 1404 bp, synthetic construct
  source 1..1404: mol_type = other DNA
  sig_peptide 1..54
atgggatggtcatgtatcatcctttttctagtagcaactgcaaccggtgtacattgccaaatcacactgaaggagagcggaccaaccctggtgaagccaacccagacactgaccctgacatgcaccgtctctggctttagcctgacaggctacggcgtgaactggatcagacagccacctggcaaggccctggagtggctgggaatgatctggggcgacggacggacagattatagccccagcctgaaatccagactgaccatcaccaaggacacctctaagaaccaggtggtgctgacaatgaccaatatggaccccgtggatacagccacctactattgcgcctcagactattacggctcaggttcgttcgcatactggggcaggggtaccctggtcacggtgtcttctgcttcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgctgcatgaggctctgcacagccactacacgcagaagagcctctccctgtccccgggtaaatga

SEQ ID NO: 25 - DNA, 1404 bp, synthetic construct
  source 1..1404: mol_type = other DNA
  sig_peptide 1..54
atgggatggtcatgtatcatcctttttctagtagcaactgcaaccggtgtacattgccaagtccaactggtggagtctggaggaggcctggtgcagccaggaggctctctgcggctgagctgctcagtcagcggcttctccctgaccggctacggagtgaactgggtgaggcaggcacctggcaagggcctggagtggctgggaatgatctggggcgacggaaggacagactacgcagatagcgtgaagggccggtttaccatctccagagacaactctaagaatacactgtatctgcagatgagcagcctgcgggccgaggataccgccgtgtactattgcgcctcagactattacggctcaggttcgttcgcatactggggccagggtaccctggtcacggtgtcttctgcttcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgctgcatgaggctctgcacagccactacacgcagaagagcctctccctgtccccgggtaaatga

SEQ ID NO: 26 - DNA, 1404 bp, synthetic construct
  source 1..1404: mol_type = other DNA
  sig_peptide 1..54
atgggatggtcatgtatcatcctttttctagtagcaactgcaaccggtgtacattgccaagtccaactggtggagtctggaggaggcctggtgcagccaggacgctctctgcggctgagctgcacagtcagcggcttctccctgaccggctacggagtgaactggttcaggcaggcacctggcaagggcctggagtggctgggaatgatctggggcgacggaaggacagactacacagctagcgtgaagggccggtttaccatctccagagacggctctaagagtatagcgtatctgcagatgaacagcctgaagaccgaggataccgccgtgtactattgcgcctcagactattacggctcaggttcgttcgcatactggggccagggtaccctggtcacggtgtcttctgcttcgaccaagggcccatcggtcttccccctggcaccctcctccaagagcacctctgggggcacagcggccctgggctgcctggtcaaggactacttccccgaacctgtgacggtctcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgctgcatgaggctctgcacagccactacacgcagaagagcctctccctgtccccgggtaaatga

SEQ ID NO: 27 - DNA, 702 bp, synthetic construct
  source 1..702: mol_type = other DNA
  sig_peptide 1..54
atgggatggtcatgtatcatcctttttctagtagcaactgcaaccggtgtacattctgatatcgtgatgacccagaccccactgtctctgtccgtgacacctggacagccagccagcatctcctgcaaggccagccaggacgtgtccaccgcagtggcctggtatctgcagaagcccggccagcctcctcagctgctgatccactctgccagctaccggtataccggcgtgcccgacagattctccggctctggcagcggcaccgacttcaccctgaagatcagccgggtggaggccgaggatgtgggcgtgtattactgccagcaacattatagcatcccgctcacgttcggtccggggaccaaggtggacatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 28 - DNA, 703 bp, synthetic construct
  source 1..703: mol_type = other DNA
  sig_peptide 1..54
atgggatggtcatgtatcatcctttttctagtagcaactgcaaccggtgtacattctgaaattgttatgacgcaatctccagcaactttgtccctttctccaggggaacgggctacgctctcttgtaaagcttcacaagatgtcagtacggcggttgcttggtatcaacagaaaccggggcaagctcctagattgctcatacattccgcgtcatatcgctatacaggaataccagcacggttttcaggatcagggtcagggacggattttaccctcacgatatcaagtcttcaaccagaagattttgcagtctattactgtcaacaacattatagtattccactcacttttggacaagggaccaaagtcgaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaga

SEQ ID NO: 29 - DNA, 702 bp, synthetic construct
  source 1..702: mol_type = other DNA
  sig_peptide 1..54
atgggatggtcatgtatcatcctttttctagtagcaactgcaaccggtgtacattctgatatcgtgatgacccagtccccagactctctcgctgtctctttgggagaaagagccaccataaactgcaaggcatcccaggacgtatcaaccgccgttgcatggtaccagcagaaacccggacagcccccaaagcttcttatacacagcgcttcttaccgatacacaggagtgcccgaccgcttcagtgggtcaggctctggaacagactttaccctgacgataagctctttgcaggcggaagacgtggccgtctactactgccagcagcactacagcatccccttaactttcggtcagggcacacgactggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 30 - protein, 467 aa, synthetic construct
  SIGNAL 1..18
MGWSCIILFLVATATGVHCQITLKESGPTLVKPTQTLTLTCTVSGFSLTGYGVNWIRQPPGKALEWLGMIWGDGRTDYSPSLKSRLTITKDTSKNQVVLTMTNMDPVDTATYYCASDYYGSGSFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

(19) United States
(12) Patent Application Publication
(10) Pub. No.: US 2025/0092158 A1
(43) Pub. Date: Mar. 20, 2025

(54) HUMANIZED ANTI QUIESCIN SUEFHYDRYE OXIDASE 1 (QSOX1) ANTIBODIES AND USES THEREOF

(71) Applicant: YEDA RESEARCH AND DEVELOPMENT CO. LTD., Rehovot (IL)

(72) Inventors: Deborah FASS, Rehovot (IL); Sarel FLEISHMAN, Rehovot (IL); Lev KHMELNITSKY, Rehovot (IL); Ariel TENNENHOUSE, Rehovot (IL)

(73) Assignee: YEDA RESEARCH AND DEVELOPMENT CO. LTD., Rehovot (IL)

(21) Appl. No.: 18/832,710

(22) PCT Filed: Aug. 4, 2022
(86) PCT No.: PCT/IL2022/050844

§ 371 Date: Jul. 24, 2024

Related U.S. Application Data
(60) Provisional application No. 63/307,166, filed on Feb. 7, 2022.

(51) Int. Cl. C07K 16/40; A61K 39/00; A61K 47/68; G01N 33/573

(52) U.S. Cl. CPC C07K 16/40; A61K 47/6871; G01N 33/573; A61K 2039/505; C07K 2317/24; C07K 2317/55; C07K 2317/565; C07K 2317/76; G01N 2333/90212

(57) ABSTRACT

The present invention provides humanized monoclonal antibodies that specifically recognize human QSOX1 and inhibit its activity. The humanized antibodies of the present invention were designed following in-silico selection of specific variable region segments, based on their energy scores, and were produced and confirmed to function properly in binding and inhibiting human QSOX1. The present invention further provides pharmaceutical compositions comprising the humanized antibodies and methods for their use in cancer therapy and diagnosis.

### FIELD OF THE INVENTION

The present invention is in the fields of immunology and immunotherapy and relates to antibodies which bind to the human protein Quiescin Sulfhydryl Oxidase 1 (QSOX1), to their fragments and conjugates, to encoding polynucleotide sequences, to constructs and cells for producing the antibodies, to pharmaceutical compositions comprising them and to their therapeutic and theragnostic uses.

### BACKGROUND OF THE INVENTION

The use of monoclonal antibodies has become an integral part of cancer diagnostics and treatment (Scott et al., Antibody therapy of cancer. Nat Rev Cancer. 2012 Mar. 22; 12(4):278-87. doi: 10.1038/nrc3236). The success of antibody-based therapy stems from the high specificity and affinity that antibodies offer compared with other antitumor agents. In addition, tumors express on their cell surfaces potential targets for antibody therapeutics. Antibodies supplied extracellularly can both neutralize the function of their cell-surface antigen and recruit the immune system for a more extensive antitumor response (Hudis, CA. Trastuzumab-mechanism of action and use in clinical practice. N Engl J Med. 2007 Jul. 5; 357(1):39-51. doi: 10.1056/NEJMra043186; Weiner, L., Monoclonal antibodies: versatile platforms for cancer immunotherapy. Nat Rev Immunol 2010, 10, 317-327. doi: 10.1038/nri2744).

The extracellular matrix (ECM) plays an important role in tumor development and dissemination, but few points of therapeutic intervention targeting ECM of the tumor microenvironment have been exploited to date. Tumor stroma has a major role in supporting tumor development and metastasis therefore antibody-based cancer therapies targeting ECM components in addition to targeting tumor cells directly was utilized. ECM proteins are good candidates for antibody therapy because they are accessible and abundant in most tissues, making the same ECM components a target in various cancers. Examples for agents that target such proteins are antibodies that affect the extracellular glycoprotein tenascin (Rizzieri et al., Phase 1 trial study of ¹³¹I-labeled chimeric 81C6 monoclonal antibody for the treatment of patients with non-Hodgkin lymphoma. Blood 2004 August 1;104(3):642-8. doi: 10.1182/blood-2003-12-4264) or fibroblast activation protein, found in stromal fibroblasts of most human carcinomas (Scott et al., A Phase I dose-escalation study of sibrotuzumab in patients with advanced or metastatic fibroblast activation protein-positive cancer. Clin Cancer Res. 2003 May; 9(5):1639-47). Inhibitors, including monoclonal antibodies, of the activity of the enzyme lysyl oxidase (LOX) in collagen cross-linking, significantly inhibited tumor growth and metastasis in gastric carcinoma (Peng et al., Secreted LOXL2 is a novel therapeutic target that promotes gastric cancer metastasis via the Src/FAK pathway, Carcinogenesis 2009, Volume 30, Issue 10, 1660-1669. doi: 10.1093/carcin/bgpl78; Rodriguez-Vita et al., The resolution of inflammation and cancer, Cytokine & Growth Factor Reviews 2010, Vol. 21, Issue 1, 61-65. doi: 10.1016/j.cytogfr.2009.11.006). Laminin, another abundant scaffolding ECM protein that interacts with integrins to mediate cell adhesion and migration, a requirement for metastasis, is overexpressed in various cancers, and its chain isotypes serve as tumor biomarkers (Kosanarn et al., Laminin, gamma 2 (LAMC2): a promising new putative pancreatic cancer biomarker identified by proteomic analysis of pancreatic adenocarcinoma tissues. Mol Cell Proteomics. 2013 October;12(10):2820-32. doi: 10.1074/ncp.M112,023507). Like collagen cross-linking and integrin blocking (Liu Z et al., Integrin alpha(v)beta(3)-Targeted Cancer Therapy. Drug Dev Res. 2008; 69(6):329-339. doi: 10.1002/ddr.20265), laminin incorporation into the matrix may serve as a complementary target for antibody-based cancer therapeutics. It has been shown that laminin incorporation into the ECM is affected by the disulfide catalyst Quiescin sulfhydryl oxidase 1 (QSOX1) (Ilani et al., A secreted disulfide catalyst controls extracellular matrix composition and function. Science. 2013 Jul. 5; 341(6141):74-6. doi: 10.1126/science.1238279). Recent observations suggest that the enzymatic introduction of disulfide bond cross-links into the ECM may be modulated to affect cancer progression. Specifically, the disulfide bond-forming activity of QSOX1 is required by fibroblasts to assemble ECM components for adhesion and migration of cancer cells.

Based on the increased protein expression of QSOX1 in the stroma of aggressive breast carcinomas, monoclonal antibody inhibitors have been developed, with the aim of preventing QSOX1 from participating in pro-metastatic ECM remodeling. Grossman et al. disclose an inhibitory mouse antibody that blocks the first step in the dithiol/disulfide relay mechanism of the enzyme QSOX1 (Grossman et al., An inhibitory antibody blocks the first step in the dithiol/disulfide relay mechanism of the enzyme QSOX1. J. Mol. Biol. 2013, 425 (22), 4366-4378). It was shown that QSOX1 inhibitory antibodies decreased tumor growth and metastasis in murine cancer models and had added benefits when provided together with chemotherapy (Feldman et al., Inhibition of fibroblast secreted QSOX1 perturbs extracellular matrix in the tumor microenvironment and decreases tumor growth and metastasis in murine cancer models. Oncotarget 2020 January 28;11(4):386-398, doi: 10.18632/oncotarget.27438). Targeting excess stromal QSOX1 secreted in response to tumor-cell signaling provides a means to modulate the tumor microenvironment and may complement other therapeutic approaches in cancer.

WO2020/035863 discloses chimeric anti-QSOX1 antibodies comprising specific sets of complementarity-determining region (CDR) sequences.

Antibody humanization is an iterative and often frustrating process that includes a first step of chimerizing the animal variable domain (Fv) with human constant domains, and a second step of humanization of the Fv. The humanization step is complicated as the CDRs, which are responsible for antigen recognition, need to be grafted from the animal source onto a human framework, typically leading to an Fv with more than 80% sequence identity to the human germline (compared to 50-70% identity for a mouse Fv). To increase the chances that the grafted CDRs are compatible with the human framework, the latter are typically picked from those showing the highest homology to the parental antibody. Other approaches to antibody humanization use structure similarity in the CDR regions rather than sequence homology, humanize only predicted immunogenic segments in the parental framework, or graft fragments from human frameworks into the animal antibody.

Despite these advances, however, Fv humanization typically leads to substantial decrease in expression levels, stability, affinity, and/or specificity. The deterioration in the antibody's biophysical properties is especially detrimental in the context of an antibody that is destined for clinical use as it leads to reduction in efficacy and can lead to undesirable complications in formulating and delivering the drug. As a rule, therefore, a third step of “backmutation” changes amino acid positions in the humanized antibody to their parental identities through iterative design-and-experiment cycles.

The underlying reason for the loss in the functional and stability parameters through humanization is structural and energetic. Structural analyses singled out the importance of a region of the framework called the vernier zone, which underlies the CDRs. Despite the relatively high conservation of the framework, the vernier zone comprises approximately 30 sequence determinants that vary even among homologous frameworks; these determinants are essential for the structural integrity and relaxation of the CDRs. Thus, most backmutation attempts use structural modeling to select mutations that reconstitute some of the vernier-zone positions seen in the animal antibody. This process can regain the parental antibody's affinity and stability, though at the cost of lower humanness and lengthy iterations.

Anti-QSOX1 inhibitory antibodies slow tumor growth and metastasis in murine breast cancer and melanoma models and therefore have potential use as human cancer therapeutics. However, prior attempts to provide effective humanized antibodies against his target were unsuccessful and currently no anti-human QSOX1 antibodies compatible with use in humans existed. There is an unmet need for stable humanized monoclonal antibodies that will specifically and effectively bind the human protein QSOX1 and inhibit its activity. These humanized antibodies may beneficially be used alone, or in combination with other agents or therapeutic approaches, for treatment of cancer.

### SUMMARY OF THE INVENTION

The present invention provides, according to some embodiments, humanized monoclonal antibodies and fragments thereof that specifically bind human Quiescin Sulfhydryl Oxidase 1 (QSOX1), and prevent its activity. Producible, stable, and highly active humanized antibodies to QSOX1 are now disclosed while earlier attempts using classical humanization methods to produce humanized antibodies to this target have failed to make expressible antibodies. The novel humanized antibodies of the present invention are disclosed for use in treatment of cancer in human subjects and for diagnostic and theragnostic uses.

The humanized antibodies of the present invention were selected from a very large collection of antibody variable region segment sequences (about 26,000 different ones), created through recombining all options of the four human germline segments (retrieved from the international immunoglobulin information system, IMGT), using a computational method that is based on structural and energy-based ranking. Each humanized antibody sequence is characterized by having one light V, one light J, one heavy V, and one heavy J human antibody germline gene segment, that includes the same set of six CDR sequences that are derived from parental murine antibody). The antibody designs of the large collection were clustered by light chain-heavy chain pair subgroups, and the highest ranked sequences (based on energy and structure) from high-scoring clusters were produced and tested for inhibition of QSOX1 activity in human cells. As a results of this combined computational and in-vitro experimental strategy, a relatively small collection of antibodies comprising specific variable regions was identified. The top-ranked designs from the energy-based humanization, identified without involving iterative backmutation processes, were well expressed, stable and showed good affinity values. Surprisingly, the experimentally best-performing humanized antibody designs identified using energy-based humanization were derived from human frameworks that are not of the highest homology with respect to the parent, murine monoclonal antibody. These antibodies are disclosed herein by their light chain and heavy chain variable domain sequences as well as by the genes used for their design (light V-light J-heavy V-heavy J).

The present invention provides, according to one aspect, stable and producible humanized antibodies that specifically bind human Quiescin Sulfhydryl Oxidase 1 (hereinafter, anti-QSOX1), or antigen-binding fragments thereof comprising at least the antigen binding domains, said antibodies or fragments thereof have inhibitory constant (Ki) of 5 nM or lower to human QSOX1. According to some embodiments, a humanized antibody according to the present invention has a Ki of 1 nm or lower, 0.5 nm or lower, 0.1 nm or lower, to human QSOX1.

According to an aspect of some embodiments of the present invention, a humanized antibody that specifically binds human QSOX1 or an antigen-binding fragment comprising at least the antigen binding domain, is provided comprising: three CDRs of a heavy-chain (HC) variable region comprising the sequence:
QVQLKQSGPGLVAPSQSLSITCTVSGFSLTGYGVNWVRQSPGKGLEWLGMIWGDGR TDYKSALKSRLSITKDNSKSQVFLKMNSLQTDDTARYFCASDYYGSGSFAYWGQGT LVTVSA (SEQ ID NO: 1); and three CDRs of a light-chain (LC) variable comprising the sequence

(SEQ ID NO: 2)
DVVMTQSHKFMSTSVGDRVSITCKASQDVSTAVAWYQQKSGQSPKLL
IHSASYRYTGVPDRFTGSGSGTDFTFTISSVQAEDLAVYYCQQHYSI
PLTFGAGTKLELK.

According to some embodiments, the complementarity-determining regions (CDRs) of SEQ ID NOs: 1 and 2 are determined using Kabat numbering to be: heavy chain CDR1 24-35B; heavy chain CDR2 47-58; heavy chain CDR3 93-103; light chain CDR1 24-34; light chain CDR2 46-55; light chain CDR3 89-97.

According to some embodiments of the present invention, the humanized anti-QSOX1 antibodies comprise a set of the 6 CDR sequences wherein, heavy chain CDR1 comprises the sequence VSGFSLTGYGVN (SEQ ID NO: 3); heavy chain CDR2 comprises the sequence WLGMIWGDGRTD (SEQ ID NO: 4); heavy chain CDR3 comprises the sequence ASDYYGSGSFAY (SEQ ID NO: 5); light chain CDR1 comprises the sequence KASQDVSTAVA (SEQ ID NO: 6); light chain CDR2 comprises the sequence LLIHSASYRY (SEQ ID NO: 7); and light chain CDR3 comprises the sequence QQHYSIPLT (SEQ ID NO: 8).

According to some embodiments of the present invention, the humanized anti-QSOX1 antibodies comprise a set of the 6 CDR sequences wherein, heavy chain CDR1 consists of the sequence VSGFSLTGYGVN (SEQ ID NO: 3); heavy chain CDR2 consists of the sequence WLGMIWGDGRTD (SEQ ID NO: 4); heavy chain CDR3 consists of the sequence ASDYYGSGSFAY (SEQ ID NO: 5); light chain CDR1 consists of the sequence KASQDVSTAVA (SEQ ID NO: 6); light chain CDR2 consists of the sequence LLIHSASYRY (SEQ ID NO: 7); and light chain CDR3 consists of the sequence QQHYSIPLT (SEQ ID NO: 8).

According to some embodiments, the humanized anti-QSOX1 antibody comprises a heavy-chain variable region selected from the group consisting of: IGHV2-5-IGHJ6 (IGHV2a_v3) having the sequence

(SEQ ID NO: 9)
QITLKESGPTLVKPTQTLTLTCTVSGFSLTGYGVNWIRQPPGKALEW
LGMIWGDGRTDYSPSLKSRLTITKDTSKNQVVLTMTNMDPVDTATYY
CASDYYGSGSFAYWGQGTTVTVSS;

IGHV2-5-IGHJ2 (IGHV2b_v3) having the sequence QITLKESGPTLVKPTQTLTLTCTVSGFSLTGYGVNWIRQPPGKALEWLGMIWGDGRT DYSPSLKSRLTITKDTSKNQVVLTMTNMDPVDTATYYCASDYYGSGSFAYWGRGTL VTVSS (SEQ ID NO: 10); and
IGHV3-64D-IGHJ1 (IGHV3_v3) having the sequence EVQLVESGGGLVQPGGSLRLSCSVSGFSLTGYGVNWVRQAPGKGLEWLGMIWGDG RTDYADSVKGRFTISRDNSKNTLYLQMSSLRAEDTAVYYCASDYYGSGSFAYWGQ GTLVTVSS (SEQ ID NO: 11); or an analog or derivative thereof having at least 90% sequence identity with any of said heavy chain variable region sequences. According to some embodiments, the antibody analog or derivative has a heavy chain variable region having at least 95% identity with any of said sequences. Each option represents a separate embodiment of the present invention.

According to some embodiments, the humanized anti-QSOX1 antibody comprises a light-chain variable region selected from the group consisting of: IGKV1-33-IGKJ1 (IGKV1a_v3) having the sequence

(SEQ ID NO: 12)
DIQMTQSPSSLSASVGDRVTITCKASQDVSTAVAWYQQKPGKAPKLL
IHSASYRYTGVPSRFSGSGSGTDFTFTISSLQPEDIATYYCQQHYSI
PLTFGQGTKVEIK;

IGKV2D-29-IGKJ3 (IGKV2_v3) having the sequence

(SEQ ID NO: 13)
DIVMTQTPLSLSVTPGQPASISCKASQDVSTAVAWYLQKPGQPPQLL
IHSASYRYTGVPDRFSGSGSGTDFTLKISRVEAEDVGVYYCQQHYSI
PLTFGPGTKVDIK;

IGKV3D-7-IGKJ1 (IGKV3_v3), having the sequence

(SEQ ID NO: 14)
EIVMTQSPATLSLSPGERATLSCKASQDVSTAVAWYQQKPGQAPRLL
IHSASYRYTGIPARFSGSGSGTDFTLTISSLQPEDFAVYYCQQHYSI
PLTFGQGTKVEIK;

IGKV4-1-IGKJ5 (IGKV4_v3) having the sequence DIVMTQSPDSLAVSLGERATINCKASQDVSTAVAWYQQKPGQPPKLLIHSASYRYTG VPDRFSGSGSGTDFTLTISSLQAEDVAVYYCQQHYSIPLTFGQGTRLEIK (SEQ ID NO: 15); and
IGKV1-13-IGKJ1 (IGKV1b_v3) having the sequence AIQLTQSPSSLSASVGDRVTITCKASQDVSTAVAWYQQKPGKAPKLLIHSASYRYTG VPSRFSGSGSGTDFTLTISSLQPEDFATYYCQQHYSIPLTFGQGTKVEIK (SEQ ID NO: 16); or an analog or derivative thereof having at least 90% sequence identity with said light chain variable region sequences. According to some embodiments, the antibody analog or derivative has a light chain variable region having at least 95% identity with any of said sequences. Each option represents a separate embodiment of the present invention.

According to some embodiments, the humanized anti-QSOX1 antibody comprises a heavy-chain variable region selected from the group consisting of: IGHV2-5-IGHJ6 (IGHV2a_v3) having the sequence

(SEQ ID NO: 9)
QITLKESGPTLVKPTQTLTLTCTVSGFSLTGYGVNWIRQPPGKALEW
LGMIWGDGRTDYSPSLKSRLTITKDTSKNQVVLTMTNMDPVDTATYY
CASDYYGSGSFAYWGQGTTVTVSS;

IGHV2-5-IGHJ2 (IGHV2b_v3) having the sequence QITLKESGPTLVKPTQTLTLTCTVSGFSLTGYGVNWIRQPPGKALEWLGMIWGDGRT DYSPSLKSRLTITKDTSKNQVVLTMTNMDPVDTATYYCASDYYGSGSFAYWGRGTL VTVSS (SEQ ID NO: 10); and
IGHV3-64D-IGHJ1 (IGHV3_v3) having the sequence EVQLVESGGGLVQPGGSLRLSCSVSGFSLTGYGVNWVRQAPGKGLEWLGMIWGDG RTDYADSVKGRFTISRDNSKNTLYLQMSSLRAEDTAVYYCASDYYGSGSFAYWGQ GTLVTVSS (SEQ ID NO: 11); and
a light-chain variable region selected from the group consisting of: IGKV1-33-IGKJ1 (IGKV1a_v3) having the sequence

(SEQ ID NO: 12)
DIQMTQSPSSLSASVGDRVTITCKASQDVSTAVAWYQQKPGKAPKLL
IHSASYRYTGVPSRFSGSGSGTDFTFTISSLQPEDIATYYCQQHYSI
PLTFGQGTKVEIK;

IGKV2D-29-IGKJ3 (IGKV2_v3) having the sequence

(SEQ ID NO: 13)
DIVMTQTPLSLSVTPGQPASISCKASQDVSTAVAWYLQKPGQPPQLL
IHSASYRYTGVPDRFSGSGSGTDFTLKISRVEAEDVGVYYCQQHYSI
PLTFGPGTKVDIK;

IGKV3D-7-IGKJ1 (IGKV3_v3), having the sequence

(SEQ ID NO: 14)
EIVMTQSPATLSLSPGERATLSCKASQDVSTAVAWYQQKPGQAPRLL
IHSASYRYTGIPARFSGSGSGTDFTLTISSLQPEDFAVYYCQQHYSI
PLTFGQGTKVEIK;

IGKV4-1-IGKJ5 (IGKV4_v3) having the sequence DIVMTQSPDSLAVSLGERATINCKASQDVSTAVAWYQQKPGQPPKLLIHSASYRYTG VPDRFSGSGSGTDFTLTISSLQAEDVAVYYCQQHYSIPLTFGQGTRLEIK (SEQ ID NO: 15); and
IGKV1-13-IGKJ1 (IGKV1b_v3) having the sequence AIQLTQSPSSLSASVGDRVTITCKASQDVSTAVAWYQQKPGKAPKLLIHSASYRYTG VPSRFSGSGSGTDFTLTISSLQPEDFATYYCQQHYSIPLTFGQGTKVEIK (SEQ ID NO: 16); or an analog or derivative thereof having at least 90% or at least 95% sequence identity with any of said heavy chain or light chain variable region sequences. Each option represents a separate embodiment of the present invention.

According to some embodiments, the humanized anti-QSOX1 antibody or the antigen-binding fragment thereof comprises a combination of human antibody germline gene segments (heavy V, heavy J, light V, and light J), wherein the heavy chain variable region gene combination is selected from the group consisting of: IGHV2-5-IGHJ6; IGHV2-5-IGHJ2; and IGHV3-64D-IGHJ1; and the light chain variable region gene combination is selected from the group consisting of: IGKV1-33-IGKJ1; IGKV2D-29-IGKJ3; IGKV3D-7-IGKJ1; IGKV4-1-IGKJ5; and IGKV1-13-IGKJ1, or an analog or derivative thereof having at least 95% sequence identity with any of said variable region segment sequences. Each option represents a separate embodiment of the present invention.

According to some embodiments, the humanized anti-QSOX1 antibody comprises heavy-chain selected from the group consisting of (the underlined represents the signal sequence):

(SEQ ID NO: 17, heavy chain H2b)
MGWSCIILFLVATATGVHCQITLKESGPTLVKPTQTLTLTCTVSGFS
LTGYGVNWIRQPPGKALEWLGMIWGDGRTDYSPSLKSRLTITKDTSK
NQVVLTMTNMDPVDTATYYCASDYYGSGSFAYWGRGTLVTVSSASTK
GPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRV
EPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVL
HQDWLNGKEYKCKVSNKALPAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVLHEALHSHYTQKSLSLSPGK;
(SEQ ID NO: 18, heavy chain H3)
MGWSCIILFLVATATGVHCQVQLVESGGGLVQPGGSLRLSCSVSGFS
LTGYGVNWVRQAPGKGLEWLGMIWGDGRTDYADSVKGRFTISRDNSK
NTLYLQMSSLRAEDTAVYYCASDYYGSGSFAYWGQGTLVTVSSASTK
GPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRV
EPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVL
HQDWLNGKEYKCKVSNKALPAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVLHEALHSHYTQKSLSLSPGK;
(SEQ ID NO: 19, heavy chain H3new)
MGWSCIILFLVATATGVHCQVQLVESGGGLVQPGRSLRLSCTVSGFS
LTGYGVNWFRQAPGKGLEWLGMIWGDGRTDYTASVKGRFTISRDGSK
SIAYLQMNSLKTEDTAVYYCASDYYGSGSFAYWGQGTLVTVSSASTK
GPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRV
EPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVL
HQDWLNGKEYKCKVSNKALPAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVLHEALHSHYTQKSLSLSPGK;
and
(SEQ ID NO: 30, heavy chain H2a)
MGWSCIILFLVATATGVHCQITLKESGPTLVKPTQTLTLTCTVSGFS
LTGYGVNWIRQPPGKALEWLGMIWGDGRTDYSPSLKSRLTITKDTSK
NQVVLTMTNMDPVDTATYYCASDYYGSGSFAYWGQGTTVTVSSASTK
GPSVFPLAPSSKSTSGGTAALGCLVKDYFPEPVTVSWNSGALTSGVH
TFPAVLQSSGLYSLSSVVTVPSSSLGTQTYICNVNHKPSNTKVDKRV
EPKSCDKTHTCPPCPAPELLGGPSVFLFPPKPKDTLMISRTPEVTCV
VVDVSHEDPEVKFNWYVDGVEVHNAKTKPREEQYNSTYRVVSVLTVL
HQDWLNGKEYKCKVSNKALPAPIEKTISKAKGQPREPQVYTLPPSRE
EMTKNQVSLTCLVKGFYPSDIAVEWESNGQPENNYKTTPPVLDSDGS
FFLYSKLTVDKSRWQQGNVFSCSVLHEALHSHYTQKSLSLSPGK;

or an analog or derivative thereof having at least 90% or at least 95% sequence identity with any of said heavy chain sequences. Each option represents a separate embodiment of the present invention.

According to some embodiments, the humanized anti-QSOX1 antibody comprises light-chain selected from the group consisting of (the underlined represents the signal sequence):

(SEQ ID NO: 20, light chain K2)
MGWSCIILFLVATATGVHSDIVMTQTPLSLSVTPGQPASISCKASQD
VSTAVAWYLQKPGQPPQLLIHSASYRYTGVPDRFSGSGSGTDFTLKI
SRVEAEDVGVYYCQQHYSIPLTFGPGTKVDIKRTVAAPSVFIFPPSD
EQLKSGTASVVCLLNNFYPREAKVQWKVDNALQSGNSQESVTEQDSK
DSTYSLSSTLTLSKADYEKHKVYACEVTHQGLSSPVTKSFNRGEC;
(SEQ ID NO: 21, light chain K4)
MGWSCIILFLVATATGVHSDIVMTQSPDSLAVSLGERATINCKASQD
VSTAVAWYQQKPGQPPKLLIHSASYRYTGVPDRFSGSGSGTDFTLTI
SSLQAEDVAVYYCQQHYSIPLTFGQGTRLEIKRTVAAPSVFIFPPSD
EQLKSGTASVVCLLNNFYPREAKVQWKVDNALQSGNSQESVTEQDSK
DSTYSLSSTLTLSKADYEKHKVYACEVTHQGLSSPVTKSFNRGEC;
and
(SEQ ID NO: 22, light chain K3)
MGWSCIILFLVATATGVHSEIVMTQSPATLSLSPGERATLSCKASQD
VSTAVAWYQQKPGQAPRLLIHSASYRYTGIPARFSGSGSGTDFTLTI
SSLQPEDFAVYYCQQHYSIPLTFGQGTKVEIKRTVAAPSVFIFPPSD
EQLKSGTASVVCLLNNFYPREAKVQWKVDNALQSGNSQESVTEQDSK
DSTYSLSSTLTLSKADYEKHKVYACEVTHQGLSSPVTKSFNRGEC;

or an analog or derivative thereof having at least 90% or at least 95% sequence identity with any of said light chain sequences. Each option represents a separate embodiment of the present invention.

According to some embodiments, the humanized anti-QSOX1 antibody comprises a heavy-chain selected from the group consisting of: SEQ ID NO: 18 (heavy chain denoted H3), and SEQ ID NO: 17 (heavy chain denoted H2b), SEQ ID NO: 19 (heavy chain denoted H3new), and a light-chain selected from the group consisting of: SEQ ID NO: 21 (light chain denoted K4) and SEQ ID NO: 20 (light chain denoted K2).

According to some specific embodiments, the humanized anti-QSOX1 comprises a pair of heavy chain and light chain, wherein the antibody is selected from the group consisting of:
i) humanized antibody denoted H3K4 comprising the heavy chain sequence set forth in SEQ ID NO: 18 (heavy chain H3), and the light chain sequence set forth in SEQ ID NO: 21 (light chain K4);
ii) humanized antibody denoted H2bK4 comprising the heavy chain sequence set forth in SEQ ID NO: 17 (heavy chain H2b), and the light chain sequence set forth in SEQ ID NO: 21 (light chain K4); and
iii) humanized antibody denoted H3newK2 comprising the heavy chain sequence set forth in SEQ ID NO: 19 (heavy chain H3new), and the light chain sequence set forth in SEQ ID NO: 20 (light chain K2);
iv) humanized antibody denoted H3newK4 comprising the heavy chain sequence set forth in SEQ ID NO: 19 (heavy chain H3new), and the light chain sequence set forth in SEQ ID NO: 21 (light chain K4);
or an analog or derivative thereof having at least 90% or at least 95% sequence identity with said antibody sequence. Each option represents a separate embodiment of the present invention.

According to some embodiments, the humanized anti-QSOX1 antibody comprises a heavy-chain having a sequence set forth in SEQ ID NO: 18 (heavy chain denoted H3), and a light-chain having a sequence set forth in SEQ ID NO: 21 (light chain denoted K4).

According to some specific embodiments, the humanized antibody or antigen-binding fragment comprises heavy chain CDR1 comprising the sequence: VSGFSLTGYGVN (SEQ ID NO: 3), heavy chain CDR2 comprising the sequence: WLGMIWGDGRTD (SEQ ID NO: 4), heavy chain CDR3 comprising the sequence: ASDYYGSGSFAY (SEQ ID NO: 5); light chain CDR1 comprising the sequence: KASQDVSTAVA (SEQ ID NO: 6), light chain CDR2 comprising the sequence: LLIHSASYRY (SEQ ID NO: 7); and light chain CDR3 comprising the sequence: QQHYSIPLT (SEQ ID NO: 8), or analog thereof comprising no more than 5% amino acid substitution, deletion and/or insertion in the hypervariable region (HVR) that consists of the six CDR sequences.

According to some specific embodiments, the humanized antibody or antigen-binding fragment comprises heavy chain CDR1 consisting of the sequence: VSGFSLTGYGVN (SEQ ID NO: 3), heavy chain CDR2 consisting of the sequence: WLGMIWGDGRTD (SEQ ID NO: 4), heavy chain CDR3 consisting of the sequence: ASDYYGSGSFAY (SEQ ID NO: 5); light chain CDR1 consisting of the sequence: KASQDVSTAVA (SEQ ID NO: 6), light chain CDR2 consisting of the sequence: LLIHSASYRY (SEQ ID NO: 7); and light chain CDR3 consisting of the sequence: QQHYSIPLT (SEQ ID NO: 8).

According to an aspect of some embodiments of the present invention, the humanized anti-QSOX1 antibody is identified using a method comprising the following steps:
i) providing a structural model or an experimental structure of an antibody having an affinity to human QSOX1 (a parental Ab) and identifying amino acid residues of at least one complementarity-determining region (CDR) in the structural model;
ii) generating all combinations of antibody variable domain sequence segments derived from a plurality of human antibody germline sequences, and replacing corresponding amino acid residues in each of the combinations with the amino acid residues of the CDR, to thereby create a library of humanized antibody sequences;
iii) threading each of the humanized antibody sequences on the structural model to thereby obtain a plurality of threaded grafted human antibody structures, and subjecting each of the threaded grafted human antibody structures to constrained energy minimization (constrained structural relaxation) to thereby obtain a plurality of relaxed grafted human antibody structures;
iv) ranking the plurality of relaxed grafted human antibody structures by an energy score;
v) clustering the plurality of relaxed grafted human antibody structures according to V/J gene families to thereby obtain an energy ranked and gene family clustered library of humanized antibody designs; and
vi) expressing at least one humanized antibody design from at least one cluster of humanized antibody designs, testing its binding to the antigen and selecting at least one humanized antibody design having an affinity to the antigen of interest, thereby obtaining the humanized antibody having an affinity to the antigen of interest.

In some embodiments, the antibody segments are selected from the group consisting of heavy chain variable (V) gene segment, light chain variable (V) gene segment, heavy chain joining (J) gene segment, light chain joining (J) gene segment, kappa gene segment, and lambda gene segment. Each option represents a separate embodiment of the present invention.

According to some embodiments, the humanized antibody identified using the above method comprises heavy chain sequence set forth in a sequence selected from SEQ ID NOs: 17, 18 and 19, or an analog or derivative thereof having at least 90% sequence identity with the heavy chain sequence. Each option represents a separate embodiment of the present invention.

According to a specific embodiment, the humanized antibody identified using the above method comprises a heavy chain having a sequence set forth in SEQ ID NO: 18, and a light chain having a sequence set forth in SEQ ID NO: 21, or an analog thereof having at least 90% sequence identity with the light and/or heavy chain sequences.

According to a specific embodiment, the humanized antibody identified using the above method comprises a heavy chain having a sequence set forth in SEQ ID NOs: 17, and a light chain having a sequence set forth in SEQ ID NO: 21, or an analog thereof having at least 90% sequence identity with the light and/or heavy chain sequences.

According to some embodiments, the humanized antibody identified using the above method comprises light chain sequence set forth in a sequence selected from SEQ ID NOs: 20 and 21, or an analog thereof having at least 90% sequence identity with the light chain variable region sequence. Each option represents a separate embodiment of the present invention.

According to a specific embodiment, the humanized antibody identified using the above method comprises a heavy chain having a sequence set forth in SEQ ID NO: 19, and a light chain having a sequence set forth in SEQ ID NO: 20, or an analog thereof having at least 90% sequence identity with the light and/or heavy chain sequences.

According to a specific embodiment, the humanized antibody identified using the above method comprises a heavy chain having a sequence set forth in SEQ ID NO: 19, and a light chain having a sequence set forth in SEQ ID NO: 21, or an analog thereof having at least 90% sequence identity with the light and/or heavy chain sequences.

According to some embodiments, the humanized antibodies or antigen-binding fragments of the present invention specifically bind to human QSOX1 and inhibit its activity.

According to some embodiments, the humanized antibodies or antibody fragments of the present invention inhibit human QSOX1 activity of oxidizing cysteine residues in proteins.

According to some embodiments, the humanized antibodies or antigen-binding fragments of the present invention inhibit human QSOX1 activity, and consequently inhibit adhesion and migration of cancer cells to and through fibroblasts from corresponding tissues.

According to some embodiments, the humanized antibodies or antigen-binding fragments of the present invention inhibit laminin incorporation into the ECM thus inhibiting tumor cell migration via laminin incorporation.

According to some embodiments, the humanized antibodies or antigen-binding fragments of the present invention prevent QSOX1 from participating in pro-metastatic ECM remodeling.

According to some embodiments, the humanized antibodies or antigen-binding fragments of the present invention are capable of modulating tumor microenvironment by targeting excess stromal QSOX1 secreted in response to tumor-cell signaling.

According to some embodiments, the humanized antibodies or antigen-binding fragments of the present invention prevent, reduce or slow tumor growth and metastasis formation or spread.

According to some embodiments, the humanized antibody or antigen-binding fragment thereof recognizes has an inhibition constant (Ki) of 10⁻⁸M or lower to human QSOX1. According to other embodiments, the humanized antibody or antibody fragment has a Ki of 10⁻⁹ (namely 1 nM), or even lower, to human QSOX1. According to some embodiments, the humanized antibody or antibody fragment has a Ki at the range of 10⁻⁹M to 10⁻¹¹M. Each possibility represents a separate embodiment of the invention.

Analogs and derivatives of the humanized antibodies and the antigen-binding fragments described above, are also within the scope of the invention as long as they retain the activity, stability and producibility properties of the parent antibody.

According to some embodiments, the analog of the humanized antibody or antigen-binding fragment thereof has at least 90% sequence identity with any of the chains of the reference antibody sequence.

According to certain embodiments, the analog or derivative of the humanized antibody or antigen-binding fragment thereof has at least 91, 92, 93, 94, 95, 96, 97, 98 or 99% sequence identity with a variable region of the reference antibody sequence. Each possibility represents a separate embodiment of the invention.

According to some embodiments, the analog or derivative has at least 95, 96, 97, 98 or 99% sequence similarity or identity with an antibody light or heavy chain variable regions described above. According to some embodiments, the analog comprises no more than one amino acid substitution, deletion, or addition to one or more CDR sequences of the hypervariable region, namely, any one of the CDR sequences set forth in SEQ ID NOs: 3-8. Each possibility represents a separate embodiment of the present invention. According to some embodiments, the amino acid substitution is a conservative substitution.

According to some embodiments, the antibody or antigen-binding fragment comprises a hypervariable region (HVR) having light and heavy chain CDR sequences defined above, in which 1, 2, 3, 4, or 5 amino acids were substituted, deleted and/or added. Each possibility represents a separate embodiment of the invention.

According to some embodiments, the humanized antibody or antigen-binding fragment comprises a HVR having light and heavy chain CDR sequences defined above, in which one amino acid was substituted. According to specific embodiments, the antibody or antibody fragment comprises a CDR as defined above, in which one amino acid was substituted.

According to some embodiments, the humanized antibody comprises a heavy chain constant region selected from the group consisting of: human IgG1, human IgG2, human IgG3 and human IgG4. Each possibility represents a separate embodiment of the present invention.

According to some embodiments the humanized antibody comprises a human IgG1 constant region.

According to some embodiments, the humanized antibody comprises a light chain constant region selected from kappa and lambda.

According to some embodiments the humanized antibody comprises a human kappa constant region.

According to some embodiments, the humanized antibody comprises a modified Fragment Crystallizable (Fc) region, namely an Fc region engineered to remove or add a specific activity to the parent Fc region. According to some embodiments, the Fc modification alter the humanized antibody binding to at least one Fc receptor. According to some embodiments, the Fc modification influence at least one effector mechanism e.g., antibody-dependent cellular cytotoxicity (ADCC), phagocytosis and production or inhibition of inflammatory cytokines. A modification of the Fc region includes but is not limited to a substitution, deletion or addition of at least one amino acid residue, and glycoengineering by removal or addition of at least one oligosaccharide (N-glycan) moiety.

According to some embodiments, an antigen-binding fragment of a humanized antibody is provided. According to a specific embodiment, the antigen-binding fragment is selected from the group consisting of: Fab, Fab′, F(ab′)₂, Fd, Fd′, Fv, dAb, single chain variable fragment (scFv), single chain antibody (scab), “diabodies”, and “linear antibodies”. Each possibility represents a separate embodiment of the present invention.

Fab fragments of the humanized antibodies of the present invention, having VL, CL, VH and CH1 domains, and variants thereof, including Fab′ fragments are also included within the scope of the present invention as well as single chain variable fragment (scFv) molecules of the humanized antibodies provide herein. These fragments are produced using methods known in the art. Fab fragments are produced, for example by proteolytic digestion. The scFv molecules comprise the antigen binding site of the antibody, from the heavy chain and light chain variable regions, expressed in one polypeptide chain. According to certain embodiments, the scFv comprises a hinge region between the two variable regions. According to specific embodiments, the scFv comprises the heavy and light chains of the humanized antibodies described herein.

According to some embodiments, a conjugate comprising the humanized antibody or antigen-binding fragment thereof as described herein is provided. According to some embodiments, the conjugate comprises a humanized antibody or an antigen-binding fragment thereof attached, directly or through a spacer or linker, to a radioactive moiety, to an identifiable moiety, or to a cytotoxic moiety. According to other embodiments, the conjugate is an antibody-drug conjugate comprising an anti-cancer moiety attached, directly or through a spacer or linker, to a humanized antibody or to an antigen-binding fragment of the preset invention.

Polynucleotide sequences encoding humanized antibodies, having high affinity and specificity for human QSOX1, as well as vectors and host cells carrying these polynucleotide sequences, are provided according to another aspect of the present invention.

According to some embodiments, a polynucleotide sequence that encodes at least one chain of a humanized antibody specific to QSOX1, or of antigen-binding fragment thereof, is provided.

According to some embodiments, a combination of polynucleotide sequences encoding the amino acid sequences of a humanized antibody heavy chain and light chain described above are provided.

According to some embodiments, the polynucleotide sequences defined above encode a molecule selected from the group consisting of: a humanized antibody, an antigen-binding fragment, or a chain thereof and a conjugate comprising said humanized antibody or antigen-binding fragment. Each possibility represents a separate embodiment of the present invention.

According to some embodiments, a polynucleotide sequence is provided encoding a humanized antibody or antigen-binding fragment, wherein the humanized antibody or antigen-binding fragment comprises a sequence set forth in any one of SEQ ID NOs: 9-22, or an analog or derivative thereof having at least 90% sequence identity with the amino acid sequence. Each possibility represents a separate embodiment of the present invention.

According to some embodiments, the polynucleotide sequence encodes a humanized antibody heavy chain variable region sequence set forth in in any of SEQ ID NOs: 9-11, or a variant thereof having at least 90% sequence identity.

According to some embodiments, the polynucleotide sequence encodes a humanized antibody light chain variable region sequence set forth in any of SEQ ID NOs: 12-16, or a variant thereof having at least 90% sequence identity.

According to some embodiments, the polynucleotide sequence encodes a humanized antibody heavy chain comprising a sequence set forth in any of SEQ ID NOs: 17-19 or a variant thereof having at least 90% sequence identity.

According to some embodiments, the polynucleotide sequence that encodes a humanized antibody heavy chain is selected from the group consisting of (the underlined represents the signal sequence):

(SEQ ID NO: 23 , denoted H2a)
atgggatggtcatgtatcatcctttttctagtagcaactgcaaccgg
tgtacattgccaaatcacactgaaggagagcggaccaaccctggtga
agccaacccagacactgaccctgacatgcaccgtctctggctttagc
ctgacaggctacggcgtgaactggatcagacagccacctggcaaggc
cctggagtggctgggaatgatctggggcgacggacggacagattata
gccccagcctgaaatccagactgaccatcaccaaggacacctctaag
aaccaggtggtgctgacaatgaccaatatggaccccgtggatacagc
cacctactattgcgcctcagactattacggctcaggttcgttcgcat
actggggccagggtaccacggtcacggtgtcttctgcttcgaccaag
ggcccatcggtcttccccctggcaccctcctccaagagcacctctgg
gggcacagcggccctgggctgcctggtcaaggactacttccccgaac
ctgtgacggtctcgtggaactcaggcgccctgaccagcggcgtgcac
accttcccggctgtcctacagtcctcaggactctactccctcagcag
cgtggtgaccgtgccctccagcagcttgggcacccagacctacatct
gcaacgtgaatcacaagcccagcaacaccaaggtggacaagagagtt
gagcccaaatcttgtgacaaaactcacacatgcccaccgtgcccagc
acctgaactcctggggggaccgtcagtcttcctcttccccccaaaac
ccaaggacaccctcatgatctcccggacccctgaggtcacatgcgtg
gtggtggacgtgagccacgaagaccctgaggtcaagttcaactggta
cgtggacggcgtggaggtgcataatgccaagacaaagccgcgggagg
agcagtacaacagcacgtaccgtgtggtcagcgtcctcaccgtcctg
caccaggactggctgaatggcaaggagtacaagtgcaaggtctccaa
caaagccctcccagcccccatcgagaaaaccatctccaaagccaaag
ggcagccccgagaaccacaggtgtacaccctgcccccatcccgggag
gagatgaccaagaaccaggtcagcctgacctgcctggtcaaaggctt
ctatcccagcgacatcgccgtggagtgggagagcaatgggcagccgg
agaacaactacaagaccacgcctcccgtgctggactccgacggctcc
ttcttcctctatagcaagctcaccgtggacaagagcaggtggcagca
ggggaacgtcttctcatgctccgtgctgcatgaggctctgcacagcc
actacacgcagaagagcctctccctgtccccgggtaaatga;
SEQ ID NO: 24, denoted H2b)
atgggatggtcatgtatcatcctttttctagtagcaactgcaaccgg
tgtacattgccaaatcacactgaaggagagcggaccaaccctggtga
agccaacccagacactgaccctgacatgcaccgtctctggctttagc
ctgacaggctacggcgtgaactggatcagacagccacctggcaaggc
cctggagtggctgggaatgatctggggcgacggacggacagattata
gccccagcctgaaatccagactgaccatcaccaaggacacctctaag
aaccaggtggtgctgacaatgaccaatatggaccccgtggatacagc
cacctactattgcgcctcagactattacggctcaggttcgttcgcat
actggggcaggggtaccctggtcacggtgtcttctgcttcgaccaag
ggcccatcggtcttccccctggcaccctcctccaagagcacctctgg
gggcacagcggccctgggctgcctggtcaaggactacttccccgaac
ctgtgacggtctcgtggaactcaggcgccctgaccagcggcgtgcac
accttcccggctgtcctacagtcctcaggactctactccctcagcag
cgtggtgaccgtgccctccagcagcttgggcacccagacctacatct
gcaacgtgaatcacaagcccagcaacaccaaggtggacaagagagtt
gagcccaaatcttgtgacaaaactcacacatgcccaccgtgcccagc
acctgaactcctggggggaccgtcagtcttcctcttccccccaaaac
ccaaggacaccctcatgatctcccggacccctgaggtcacatgcgtg
gtggtggacgtgagccacgaagaccctgaggtcaagttcaactggta
cgtggacggcgtggaggtgcataatgccaagacaaagccgcgggagg
agcagtacaacagcacgtaccgtgtggtcagcgtcctcaccgtcctg
caccaggactggctgaatggcaaggagtacaagtgcaaggtctccaa
caaagccctcccagcccccatcgagaaaaccatctccaaagccaaag
ggcagccccgagaaccacaggtgtacaccctgcccccatcccgggag
gagatgaccaagaaccaggtcagcctgacctgcctggtcaaaggctt
ctatcccagcgacatcgccgtggagtgggagagcaatgggcagccgg
agaacaactacaagaccacgcctcccgtgctggactccgacggctcc
ttcttcctctatagcaagctcaccgtggacaagagcaggtggcagca
ggggaacgtcttctcatgctccgtgctgcatgaggctctgcacagcc
actacacgcagaagagcctctccctgtccccgggtaaatga;
(SEQ ID NO: 25, denoted H3)
atgggatggtcatgtatcatcctttttctagtagcaactgcaaccgg
tgtacattgccaagtccaactggtggagtctggaggaggcctggtgc
agccaggaggctctctgcggctgagctgctcagtcagcggcttctcc
ctgaccggctacggagtgaactgggtgaggcaggcacctggcaaggg
cctggagtggctgggaatgatctggggcgacggaaggacagactacg
cagatagcgtgaagggccggtttaccatctccagagacaactctaag
aatacactgtatctgcagatgagcagcctgcgggccgaggataccgc
cgtgtactattgcgcctcagactattacggctcaggttcgttcgcat
actggggccagggtaccctggtcacggtgtcttctgcttcgaccaag
ggcccatcggtcttccccctggcaccctcctccaagagcacctctgg
gggcacagcggccctgggctgcctggtcaaggactacttccccgaac
ctgtgacggtctcgtggaactcaggcgccctgaccagcggcgtgcac
accttcccggctgtcctacagtcctcaggactctactccctcagcag
cgtggtgaccgtgccctccagcagcttgggcacccagacctacatct
gcaacgtgaatcacaagcccagcaacaccaaggtggacaagagagtt
gagcccaaatcttgtgacaaaactcacacatgcccaccgtgcccagc
acctgaactcctggggggaccgtcagtcttcctcttccccccaaaac
ccaaggacaccctcatgatctcccggacccctgaggtcacatgcgtg
gtggtggacgtgagccacgaagaccctgaggtcaagttcaactggta
cgtggacggcgtggaggtgcataatgccaagacaaagccgcgggagg
agcagtacaacagcacgtaccgtgtggtcagcgtcctcaccgtcctg
caccaggactggctgaatggcaaggagtacaagtgcaaggtctccaa
caaagccctcccagcccccatcgagaaaaccatctccaaagccaaag
ggcagccccgagaaccacaggtgtacaccctgcccccatcccgggag
gagatgaccaagaaccaggtcagcctgacctgcctggtcaaaggctt
ctatcccagcgacatcgccgtggagtgggagagcaatgggcagccgg
agaacaactacaagaccacgcctcccgtgctggactccgacggctcc
ttcttcctctatagcaagctcaccgtggacaagagcaggtggcagca
ggggaacgtcttctcatgctccgtgctgcatgaggctctgcacagcc
actacacgcagaagagcctctccctgtccccgggtaaatga;
and
(SEQ ID NO: 26, denoted H3new)
atgggatggtcatgtatcatcctttttctagtagcaactgcaaccgg
tgtacattgccaagtccaactggtggagtctggaggaggcctggtgc
agccaggacgctctctgcggctgagctgcacagtcagcggcttctcc
ctgaccggctacggagtgaactggttcaggcaggcacctggcaaggg
cctggagtggctgggaatgatctggggcgacggaaggacagactaca
cagctagcgtgaagggccggtttaccatctccagagacggctctaag
agtatagcgtatctgcagatgaacagcctgaagaccgaggataccgc
cgtgtactattgcgcctcagactattacggctcaggttcgttcgcat
actggggccagggtaccctggtcacggtgtcttctgcttcgaccaag
ggcccatcggtcttccccctggcaccctcctccaagagcacctctgg
gggcacagcggccctgggctgcctggtcaaggactacttccccgaac
ctgtgacggtctcgtggaactcaggcgccctgaccagcggcgtgcac
accttcccggctgtcctacagtcctcaggactctactccctcagcag
cgtggtgaccgtgccctccagcagcttgggcacccagacctacatct
gcaacgtgaatcacaagcccagcaacaccaaggtggacaagagagtt
gagcccaaatcttgtgacaaaactcacacatgcccaccgtgcccagc
acctgaactcctggggggaccgtcagtcttcctcttccccccaaaac
ccaaggacaccctcatgatctcccggacccctgaggtcacatgcgtg
gtggtggacgtgagccacgaagaccctgaggtcaagttcaactggta
cgtggacggcgtggaggtgcataatgccaagacaaagccgcgggagg
agcagtacaacagcacgtaccgtgtggtcagcgtcctcaccgtcctg
caccaggactggctgaatggcaaggagtacaagtgcaaggtctccaa
caaagccctcccagcccccatcgagaaaaccatctccaaagccaaag
ggcagccccgagaaccacaggtgtacaccctgcccccatcccgggag
gagatgaccaagaaccaggtcagcctgacctgcctggtcaaaggctt
ctatcccagcgacatcgccgtggagtgggagagcaatgggcagccgg
agaacaactacaagaccacgcctcccgtgctggactccgacggctcc
ttcttcctctatagcaagctcaccgtggacaagagcaggtggcagca
ggggaacgtcttctcatgctccgtgctgcatgaggctctgcacagcc
actacacgcagaagagcctctccctgtccccgggtaaatga;

or a variant thereof having at least 90% sequence identity. Each possibility represents a separate embodiment of the present invention.

According to some embodiments, the polynucleotide sequence encodes a humanized antibody light chain comprising a sequence set forth in any of SEQ ID NOS: 20-22, or a variant thereof having at least 90% sequence identity.

According to some embodiments, the polynucleotide sequence that encodes a humanized antibody light chain is selected from the group consisting of (the underlined represents a signal sequence):

(SEQ ID NO: 27, denoted K2)
atgggatggtcatgtatcatcctttttctagtagcaactgcaaccgg
tgtacattctgatatcgtgatgacccagaccccactgtctctgtccg
tgacacctggacagccagccagcatctcctgcaaggccagccaggac
gtgtccaccgcagtggcctggtatctgcagaagcccggccagcctcc
tcagctgctgatccactctgccagctaccggtataccggcgtgcccg
acagattctccggctctggcagcggcaccgacttcaccctgaagatc
agccgggtggaggccgaggatgtgggcgtgtattactgccagcaaca
ttatagcatcccgctcacgttcggtccggggaccaaggtggacatca
aacgtacggtggctgcaccatctgtcttcatcttcccgccatctgat
gagcagttgaaatctggaactgcctctgttgtgtgcctgctgaataa
cttctatcccagagaggccaaagtacagtggaaggtggataacgccc
tccaatcgggtaactcccaggagagtgtcacagagcaggacagcaag
gacagcacctacagcctcagcagcaccctgacgctgagcaaagcaga
ctacgagaaacacaaagtctacgcctgcgaagtcacccatcagggcc
tgagctcgcccgtcacaaagagcttcaacaggggagagtgttag;
(SEQ ID NO: 28, denoted K3)
atgggatggtcatgtatcatcctttttctagtagcaactgcaaccgg
tgtacattctgaaattgttatgacgcaatctccagcaactttgtccc
tttctccaggggaacgggctacgctctcttgtaaagcttcacaagat
gtcagtacggcggttgcttggtatcaacagaaaccggggcaagctcc
tagattgctcatacattccgcgtcatatcgctatacaggaataccag
cacggttttcaggatcagggtcagggacggattttaccctcacgata
tcaagtcttcaaccagaagattttgcagtctattactgtcaacaaca
ttatagtattccactcacttttggacaagggaccaaagtcgaaatta
aacgtacggtggctgcaccatctgtcttcatcttcccgccatctgat
gagcagttgaaatctggaactgcctctgttgtgtgcctgctgaataa
cttctatcccagagaggccaaagtacagtggaaggtggataacgccc
tccaatcgggtaactcccaggagagtgtcacagagcaggacagcaag
gacagcacctacagcctcagcagcaccctgacgctgagcaaagcaga
ctacgagaaacacaaagtctacgcctgcgaagtcacccatcagggcc
tgagctcgcccgtcacaaagagcttcaacaggggagagtgttaga;
(SEQ ID NO: 29, denoted K4)
atgggatggtcatgtatcatcctttttctagtagcaactgcaaccgg
tgtacattctgatatcgtgatgacccagtccccagactctctcgctg
tctctttgggagaaagagccaccataaactgcaaggcatcccaggac
gtatcaaccgccgttgcatggtaccagcagaaacccggacagccccc
aaagcttcttatacacagcgcttcttaccgatacacaggagtgcccg
accgcttcagtgggtcaggctctggaacagactttaccctgacgata
agctctttgcaggcggaagacgtggccgtctactactgccagcagca
ctacagcatccccttaactttcggtcagggcacacgactggagatca
aacgtacggtggctgcaccatctgtcttcatcttcccgccatctgat
gagcagttgaaatctggaactgcctctgttgtgtgcctgctgaataa
cttctatcccagagaggccaaagtacagtggaaggtggataacgccc
tccaatcgggtaactcccaggagagtgtcacagagcaggacagcaag
gacagcacctacagcctcagcagcaccctgacgctgagcaaagcaga
ctacgagaaacacaaagtctacgcctgcgaagtcacccatcagggcc
tgagctcgcccgtcacaaagagcttcaacaggggagagtgttag;

or a variant thereof having at least 90% sequence identity. Each possibility represents a separate embodiment of the present invention.

In a further aspect, the present invention provides a nucleic acid construct comprising a nucleic acid molecule encoding at least one chain of a humanized antibody or antigen-binding fragment thereof as described herein. According to some embodiments the nucleic acid construct is a plasmid.

According to some embodiments, the construct comprises a polynucleotide sequence selected from the group consisting of SEQ ID NO: 23, SEQ ID NO: 24, SEQ ID NO: 25, and SEQ ID NO: 26, or a variant thereof having at least 90% sequence identity, encoding a heavy chain and a polynucleotide sequence selected from the group consisting of SEQ ID NO: 27, SEQ ID NO: 28 and SEQ ID NO: 29, or a variant thereof having at least 90% sequence identity, encoding a light chain. Each possibility represents a separate embodiment of the present invention.

According to some embodiments, the construct comprises a polynucleotide sequence selected from the group consisting of SEQ ID NO: 26, SEQ ID NO: 25 and SEQ ID NO: 24, or a variant thereof having at least 90% sequence identity, encoding a heavy chain and a polynucleotide sequence selected from the group consisting of SEQ ID NO: 27 and SEQ ID NO: 29, or a variant thereof having at least 90% sequence identity, encoding a light chain. Each possibility represents a separate embodiment of the present invention.

In still another aspect the present invention provides a host cell or a population or a culture of host cells, comprising at least one of the above polynucleotide sequences, wherein these cells are capable of producing a humanized antibody or an antigen-binding fragment thereof that comprises the specific heavy and light chain variable regions described herein.

According to some embodiments, the present invention provides a construct or a cell comprising at least one construct, capable of producing a humanized antibody specific to human QSOX1, wherein the humanized antibody is selected from the group consisting of H3K4, H3newK2, H3newK4, and H2bK4 defined above.

The present invention provides, according to some embodiments, a polypeptide comprising at least one sequence encoded by at least one polynucleotide sequence disclosed above.

The present invention provides, according to another aspect, a pharmaceutical composition comprising as an active ingredient, at least one humanized antibody that specifically binds QSOX1, or an antigen-binding fragment or a conjugate thereof, and optionally at least one pharmaceutical acceptable excipient, diluent, salt, or carrier.

According to some embodiments, the pharmaceutical composition comprises at least one humanized antibody that specifically binds QSOX1, or an antigen-binding fragment or a conjugate thereof described above, and at least one pharmaceutical acceptable excipient, diluent, salt, or carrier.

According to some embodiments, the pharmaceutical composition comprises at least one humanized antibody or antigen-binding fragment thereof comprising a set of six CDRs wherein: heavy chain CDR1 is VSGFSLTGYGVN (SEQ ID NO: 3); heavy chain CDR2 is WLGMIWGDGRTD (SEQ ID NO: 4); heavy chain CDR3 is ASDYYGSGSFAY (SEQ ID NO: 5); light chain CDR1 is KASQDVSTAVA (SEQ ID NO: 6); light chain CDR2 is LLIHSASYRY (SEQ ID NO: 7); and light chain CDR3 is QQHYSIPLT (SEQ ID NO: 8).

According to some embodiments, the pharmaceutical composition comprises a humanized antibody or antigen-binding fragment thereof, comprising a heavy-chain variable region sequence selected from the group consisting of SEQ ID NO: 9-11. Each possibility represents a separate embodiment of the invention.

According to some embodiments, the pharmaceutical composition comprises a humanized antibody or antigen-binding fragment thereof comprising a light chain variable region having a sequence selected from the group consisting of SEQ ID NOS: 12-16. Each possibility represents a separate embodiment of the invention.

According to a specific embodiment, the pharmaceutical composition comprises a humanized antibody or antigen-binding fragment thereof comprising a heavy chain having a sequence selected from SEQ ID NOS: 17-19 and a light chain having the sequence selected from the group consisting of SEQ ID NOS: 20-22. Each possibility represents a separate embodiment of the invention.

The pharmaceutical compositions of this invention are formulated for administration by any means suitable for antibodies. According to some embodiments, the pharmaceutical compositions are formulated for parenteral administration. According to some embodiments, the pharmaceutical compositions are formulated for intravenous (i.v.) administration. In some embodiments, the pharmaceutical compositions are formulated for administration by injection or by infusion.

Also provided are pharmaceutical compositions, comprising at least one humanized antibody, antigen-binding fragment, or antibody conjugate according to the invention, for use in inhibiting the activity of human QSOX1.

According to some embodiments, the pharmaceutical composition comprising at least one humanized antibody or antigen-binding fragment to QSOX1 is for use in inhibiting adhesion and migration of cancer cells to and through fibroblasts from corresponding tissues.

According to some embodiments, the pharmaceutical composition comprising at least one humanized antibody or antigen-binding fragment to QSOX1 is for use in inhibiting tumor cell migration via laminin incorporation.

According to some embodiments, the pharmaceutical composition comprising at least one humanized antibody or antigen-binding fragment to QSOX1 is for use in preventing, delaying or inhibiting pro-metastatic ECM remodeling.

According to some embodiments, the pharmaceutical composition comprising at least one humanized antibody or antigen-binding fragment to QSOX1 is for use in modulating tumor microenvironment by targeting excess stromal QSOX1 secreted in response to tumor-cell signaling.

According to some embodiments, the pharmaceutical composition comprising at least one humanized antibody or antigen-binding fragment to QSOX1 is for use in delaying, slowing or preventing tumor growth and metastasis formation or spread.

According to some embodiments, the pharmaceutical composition according to the present invention is for use in treatment of a laminin-associated disease or condition.

According to some embodiments, the laminin-associated disease or condition is a tumor or a cancer.

According to some embodiments, the pharmaceutical composition according to the present invention is for use in treatment of cancer.

According to some embodiments, the pharmaceutical composition is for treatment of a solid tumor.

According to other embodiments, the pharmaceutical composition is for treatment of a metastatic tumor or cancer.

According to other embodiments, the pharmaceutical composition is for treatment of a solid metastatic tumor.

According to yet other embodiments, the cancer is an advanced or metastatic fibroblast activation protein-positive cancer.

According to some specific embodiments, the solid cancer is selected from the group consisting of breast cancer and melanoma.

According to some embodiments, the tumor is adenocarcinoma. According to some embodiments, the tumor is breast adenocarcinoma.

According to some embodiments of the invention, the cancer is selected from the group consisting of a lung cancer, a breast cancer, a colorectal cancer, a melanoma, an ovarian cancer, a pancreatic cancer, a colon cancer, a cervical cancer, a kidney cancer, a thyroid cancer, a prostate cancer, a brain cancer, a renal cancer, a throat cancer, a laryngeal carcinoma, a bladder cancer, a hepatic cancer, a fibrosarcoma, an endometrial cell cancer, a glioblastoma, sarcoma, an urachus cancer, a vaginal cancer, an esophagus cancer, a stomach cancer, a leukemia, and a lymphoma. Each possibility represents a separate embodiment of the invention.

According to some embodiments, the cancer is selected from the group consisting of a prostate cancer, a lung cancer, a breast cancer, a cervical cancer, an urachus cancer, a vaginal cancer, a colon cancer, an esophagus cancer, a pancreatic cancer, a throat cancer, a stomach cancer, and a myeloid leukemia. Each possibility represents a separate embodiment of the invention.

According to yet another aspect, the present invention provides methods of inhibiting the activity of human QSOX1 in a subject in need thereof, comprising administering to said subject a therapeutically effective amount of a humanized antibody or antigen-binding fragment or conjugate thereof or a pharmaceutical composition comprising them, as defined herein.

According to yet another aspect, the present invention provides a method of treating a laminin-associated disease or condition comprising administering to a subject in need thereof, a pharmaceutical composition comprising a therapeutically effective amount of the humanized anti-QSOX1 antibody or antigen-binding fragment or conjugate thereof described herein.

According to some embodiments, the laminin-associated disease or condition is a tumor or a cancer.

According to some embodiments of the invention, the therapeutically effective amount results in a decrease in tumor size or in the formation or spread of metastases in the subject.

According to some embodiments, the cancer is a solid tumor.

According to other embodiments, the cancer is a metastatic tumor or cancer.

According to yet other embodiments, the cancer is an advanced or metastatic fibroblast activation protein-positive cancer.

According to other embodiments, the cancer is a solid metastatic tumor.

According to some specific embodiments, the solid cancer is selected from the group consisting of breast cancer and melanoma.

According to some embodiments, the tumor is adenocarcinoma. According to some embodiments, the tumor is breast adenocarcinoma.

According to yet other embodiments, the cancer is a hematological cancer selected from leukemia, lymphoma, and multiple myeloma.

According to some embodiments of the invention, the cancer is selected from the group consisting of a lung cancer, a breast cancer, a colorectal cancer, a melanoma, an ovarian cancer, a pancreatic cancer, a colon cancer, a cervical cancer, a kidney cancer, a thyroid cancer, a prostate cancer, a brain cancer, a renal cancer, a throat cancer, a laryngeal carcinoma, a bladder cancer, a hepatic cancer, a fibrosarcoma, an endometrial cells cancer, a glioblastoma, sarcoma, an urachus cancer, a vaginal cancer, an esophagus cancer, a stomach cancer, a leukemia, and a lymphoma. Each possibility represents a separate embodiment of the invention.

According to some embodiments, the cancer is selected from the group consisting of a prostate cancer, a lung cancer, a breast cancer, a cervical cancer, an urachus cancer, a vaginal cancer, a colon cancer, an esophagus cancer, a pancreatic cancer, a throat cancer, a stomach cancer, and a myeloid leukemia. Each possibility represents a separate embodiment of the invention.

The humanized antibodies, antigen-binding fragments or conjugates thereof may be administered by any means suitable for administration of antibodies, including but not limited to intravenous, intertumoral, subcutaneous, intramuscular, intranasal, intra-arterial, topical, intraarticular, intralesional, and parenteral modes. According to some embodiments, the humanized antibodies, antigen-binding fragments or conjugates are administered by intravenous (i.v.) administration. In some embodiments, the pharmaceutical compositions are formulated for administration by injection or by infusion.

According to some embodiments, the method of treating a cancer or a tumor in a subject in need of such a treatment comprises administering or performing at least one additional anti-cancer therapy. According to certain embodiments, the additional anti-cancer therapy is selected from surgery, chemotherapy, radiotherapy, and immunotherapy.

According to some embodiments, the method of treating cancer comprises administration of the humanized antibody or an antigen-binding fragment or conjugate thereof, and an additional anti-cancer agent. According to some embodiments, the additional anti-cancer agent is selected from the group consisting of: immune-modulator, an agent that bind a tumor antigen or a receptor over-expressed on tumor cells, and chemotherapeutic agent.

According to some embodiments, the additional immune-modulator is an antibody against an immune checkpoint molecule. According to some embodiments, the additional immune modulator is an antibody against an immune checkpoint molecule selected from the group consisting of programmed cell death protein 1 (PD-1), programmed cell death protein-ligand 1 (PD-L1) and programmed cell death protein-ligand 2 (PD-L2), carcinoembryonic antigen-related cell adhesion molecule 1 (CEACAM1), lymphocyte activation gene 3 (LAG3), CD137, OX40 (also referred to as CD134), killer cell immunoglobulin-like receptors (KIR), T cell immunoreceptor with Ig and ITIM domains (TIGIT), poliovirus receptor (PVR), cytotoxic T-lymphocyte associated protein 4 (CTLA-4), NKG2A, Glucocorticoid-induced TNFR-related protein (GITR), T cell immunoglobulin and mucin domain-containing protein 3 (TIM3), B and T lymphocyte attenuator (BTLA), V-domain Ig suppressor of T cell activation (VISTA), B7H4, CD96, BY55 (CD 160), Leukocyte Associated Immunoglobulin Like Receptor 1 (LAIR1), Sialic Acid Binding Ig Like Lectin 10 (SIGLEC10), and 2B4 and any other checkpoint molecule or a combination thereof. Each possibility represents a separate embodiment of the invention.

According to some embodiments, the anti-cancer agent is selected from the group consisting of: erbitux, cytarabine, fludarabine, fluorouracil, mercaptopurine, methotrexate, thioguanine, gemcitabine, vincristine, vinblastine, vinorelbine, carmustine, lomustine, chlorambucil, cyclophosphamide, cisplatin, carboplatin, ifosfamide, mechlorethamine, melphalan, thiotepa, dacarbazine, bleomycin, dactinomycin, daunorubicin, doxorubicin, idarubicin, mitomycin, mitoxantrone, plicamycin, etoposide, teniposide and any combination thereof. Each possibility represents a separate embodiment of the invention.

According to some embodiments of the invention, the subject is a human subject.

According to some embodiments, the method of treating cancer or tumor in a subject in need thereof results in preventing or reducing formation, growth or spread of metastases in the subject.

The present invention further comprises, according to another aspect, a method of determining or quantifying the presence of human QSOX1 in a biological sample, the method comprising contacting a biological sample with a humanized antibody or antigen-binding fragment or conjugate thereof detailed above, and measuring the level of complex formation.

According to some embodiments, the method is for visualizing a tumor in a subject body.

According to some embodiments, the biological sample is a body fluid or a body tissue sample. According to some embodiments, the biological sample is a biopsy taken from a subject. According to some embodiments, the humanized antibody or antigen-binding fragment used in the method is labeled with a detectable moiety, such as but not limited to a radioactive moiety and a fluorescent label.

The humanized antibodies according to the present invention, their antigen-binding fragments or conjugates, may also be used to configure screening methods. For example, an enzyme-linked immunosorbent assay (ELISA), or a radioimmunoassay (RIA), as well as methods such as immunohistochemistry (IHC) or fluorescence-activated cell sorting (FACS), can be constructed for measuring levels of secreted or cell-associated QSOX1 using the antibodies and methods known in the art.

According to some embodiments, the method is performed in-vitro or ex-vivo.

Targeted imaging may be also performed with the antibodies of the present invention or with their antigen-binding fragments or conjugates. According to some embodiments, antibodies labeled with a detectable moiety are used for cancer detection by identifying and localizing suspicious lesions and as a guide for tissue biopsy and surgical resection. According to some embodiments, targeted imaging using the antibodies of the present invention allow early detections of cancerous tissue or cells that may not otherwise be seen. Targeted imaging may also be used, according to the present invention, to determine the best choice of therapy and to monitor efficacy. These method are performed, according to some embodiments, in vivo or ex-vivo.

According to an aspect, the present invention provides a method of diagnosing, staging or prognosing cancer, cancer recurrence, disease aggressiveness or a method of theragnosis in a subject, the method comprising determining the expression level of human QSOX1 in a biological sample of said subject using at least one humanized antibody or antigen-binding fragment or conjugate thereof as described herein.

Methods for producing and purifying the humanized antibodies of the present invention, or the antigen-binding fragments, are also included within its scope. Any method known in the art to produce, isolate and purify recombinant antibodies may be used.

According to some embodiments, the method of producing a humanized antibody to QSOX1, comprises:
(a) culturing cells which comprise polynucleotide(s) of the invention under conditions which allow for expression of the VH and/or VL chains; and
(b) recovering the VH and/or VL chains from the cells.

According to other embodiments, the antibodies are assembled in the cells, secreted to the cell culture supernatant and then recovered.

According to some embodiments, the humanized antibodies are produced by transient transfection of plasmids comprising polynucleotide sequences encoding heavy and light chain pairs, into cells. Any method known in the art to recombinantly produce antibodies may be used to produce the antibodies of the present invention.

Transformed cells are cultured under effective conditions, which allow for the expression of high amounts of recombinant polypeptide. Effective culture conditions include, but are not limited to, effective media, bioreactor, temperature, pH and oxygen conditions that permit protein production. An effective medium refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins. In some embodiments, the effective medium includes serum or at least one serum protein.

Cells of the present invention can be cultured in conventional fermentation bioreactors, shake flasks, test tubes, microtiter dishes and petri plates. Culturing can be carried out at a temperature, pH and oxygen content appropriate for a recombinant cell. Such culturing conditions are within the expertise of one of ordinary skill in the art.

Depending on the vector and host system used for production, resultant antibody polypeptides of the present invention may either remain within the recombinant cell, secreted into the fermentation medium, secreted into a space between two cellular membranes; or retained on the outer surface of a cell or viral membrane.

Following a predetermined time in culture, recovery of the recombinant humanized antibody or antibody fragment is effected, e.g., by collecting the whole fermentation medium containing the humanized antibody polypeptide/s, with or without additional steps of separation or purification.

If the protein is expressed in the cell, the cell membrane is preferably disrupted so as to release the polypeptide, using methods known in the art including homogenization.

Notwithstanding the above, humanized antibodies or fragments thereof, of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, protein A/G/L separation, mix mode chromatography, metal affinity chromatography, Lectins affinity chromatography, chromatofocusing and differential solubilization.

According to some embodiments, separation and purification comprise one or more of the steps: collecting the culture medium, centrifuged to pellet cells and any particulate matter, filtration, purification using chromatography and buffer exchange.

According to some embodiments, the chromatography is a protein G chromatography.

According to a specific embodiment, following purification the humanized antibody or antibody fragment is purified to a level of at least about 95%, 96%, 97%, 98%, 99% or even higher level of purity (reflecting less than about 5% host cell contaminants w/w or w/v).

An article of manufacture comprising a humanized antibody or an antigen-binding fragment or conjugate thereof, being packaged in a packaging material, and identified in print, in or on said packaging material is also provided according to yet another aspect of the present invention.

According to some embodiments, the article of manufacture is for use in the treatment of a laminin-associated disease or condition, e.g., a tumor or a cancer. In other embodiments, the article of manufacture is for use in imaging cancer or a tumor or for guided surgery.

According to some embodiments, the article of manufacture, further comprises a chemotherapeutic agent or a biological anti-cancer agent.

According to yet another aspect, the invention provides kits comprising one or more compositions disclosed herein. In some embodiments, the invention provides kits useful for methods disclosed herein. For example, a kit may include a container having a sterile reservoir that houses any composition disclosed herein. In some embodiments, the kit further includes instructions. For example, a kit may include the instructions for administering the composition to a subject (e.g., indication, dosage, methods etc.). In yet another example the kit may include instructions regarding application of the compositions and methods of the invention to imaging systems. According to some specific embodiments, a kit for measuring the expression or presence of human QSOX1 in biological sample is provided comprising at least one humanized antibody or antigen-binding fragment or conjugate according to the present invention.

Any embodiment disclosed herein above can optionally be combined with the subject matter of one or any combination of another embodiment disclosed herein. Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a schematic representation of the computational method used to design and select the humanized antibodies of the present invention, i.e., to find which sequences best support the CDRs. Approximately 20,000 unique humanized sequences were generated for a parental antibody (anti human QSOX1 in the present invention) by replacing the variable fragment CDRs of the human germline sequences with the CDRs of the parental murine antibody. Each CDR-grafted germline sequence is then threaded onto the relaxed Fv crystal structure and the entire sequence is then ranked according to energy and clustered according to V gene subgroup. The final step is expression of at least one humanized antibody design from at least one cluster, and selection of at least one antibody design with an affinity to the antigen of interest (human QSOX1 in the present invention), thereby obtaining the energetically optimal antibody design for a specific target.

FIG. 2 shows initial expression- and activity-test of the 15 humanized anti-QSOX1 antibody designs. Each antibody is a combination of a heavy chain (denoted H2a, H2b or H3) and a light chain (K1a, K1b, K2, K3 or K4). Chimeric antibody Chim18 was used as a positive control.

FIGS. 3A and 3B depict activity test on purified antibody designs. The antibody designs that showed most expression and activity in the initial test (FIG. 2) were expressed in larger scale and purified by protein G affinity chromatography in three separate batches, FIG. 3A first and second batch, FIG. 3B. third batch). ZG16 oxidation reactions containing 50 nM QSOX1 and 50 nM antibody were performed. Four variants show high activity: H2bK4, H3K4, H3newK4 and H3newK2.

FIGS. 4A-4F depict relative change in dissolved [O₂] as a measure of QSOX1 activity on dithiothreitol (DTT) with and without murine antibody 492 (FIG. 4A), chimeric antibody (FIG. 4D) or humanized antibodies H3newK2 (FIG. 4B), H3newK4 (FIG. 4C), H3K3 (FIG. 4E) and H2bK4 (FIG. 4F). QSOX1 concentration was 25 nM and relative activity was calculated for antibody concentrations 1-100 nM.

FIG. 5 represents the Ki values calculated for the different antibodies: parental murine antibody 492, chimera, and humanized antibodies H2bK4, H3K4, H2newK2, H3newK4, using the equation detailed in Example 2.

FIGS. 6A-6C represent the fraction inhibition for the various antibodies (parental murine antibody 492, chimera, and humanized antibodies H2bK4 and H3K4) at molar ratios of antibody:QSOX1 of 2:1, 1:1, and 0.4:1. Absolute QSOX1 (25 nM) and antibody concentrations (50 nM FIG. 6A, 25 nM FIG. 6B, and 10 nM FIG. 6C) are indicated on the plots. An arrow points out substoichiometric inhibition by the H3K4 humanized antibody comparable to the parental (492) and chimeric antibodies.

FIGS. 7A and 7B are bar graphs showing blood plasma QSOX1 inhibition from two donors by the humanized antibody design H3K4 and by the parental murine antibody MAb492.1. Assays were performed by spiking samples after the indicated incubation times with DTT, allowing endogenous plasma QSOX1 to oxidize thiols at room temperature for half an hour, and then reacting the remaining thiols with the colorimetric reagent DTNB (absorbance maximum 412 nm). “-Ab” indicates samples to which no antibody was added, “-Q” indicates samples without plasma, i.e., without QSOX1. The other samples contained plasma and a final concentration of 25 nM humanized anti-QSOX1 antibody H3K4 or parental murine hybridoma antibody Mab492.1 (492). FIG. 7A donor 1. FIG. 7B donor 2.

### DETAILED DESCRIPTION OF THE INVENTION

The present invention provides effective humanized antibodies, and antigen-binding fragments and conjugates thereof, specific to the human protein QSOX1. The invention also provides compositions and uses of the humanized antibodies as therapeutic and diagnostic agents.

The selected humanized antibodies of the present invention are characterized by being composed of specific combinations of heavy chain and light chain gene segments and a single set of six CDR sequences derived from a murine parental antibody. Selected humanized antibodies having these characteristics are shown herein to be stable and reproducible, and to have high affinity and high specificity to human QSOX1. These properties make the humanized antibodies of the present invention valuable candidates for use in anti-cancer therapy, as either a stand-alone therapy or in combination with other anti-cancer agents and therapies.

It is the first time that producible and stable humanized antibodies specific to this human protein involved in ECM remodeling, cancer and metastatic spread, are disclosed. Being humanized, these antibodies avoid the risk of adverse immune response towards the antibodies and are therefore expected to be safe for in-vivo use in humans.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “plurality” means “two or more”, unless expressly specified otherwise.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

As used herein, the term “about” when combined with a value refers to plus and minus 10% of the reference value, namely that an acceptable error range, e.g., up to 10%, or up to 5% in some cases, for the particular value should be assumed.

Any concentration ranges, percentage range, or ratio range recited herein are to be understood to include concentrations, percentages or ratios of any integer within that range and fractions thereof, such as one tenth and one hundredth of an integer, unless otherwise indicated.

### Human QSOX1 Structure, Activity and Antibodies

The term “human QSOX1” as used herein refers to the human protein Quiescin Sulfhydryl Oxidase 1, defined by protein or gene encoding it, set forth for example, in SwissProt, UniProt (000391) and GenBank symbols or accession numbers: NP_001004128.1 and NP_002817.2 representing exemplary reference sequence (refseq) entries for two different splice forms of QSOX1. Other variants of this human protein are also included in the scope of the present invention as targets for humanized antibodies.

The enzyme QSOX1 is a fusion of two thioredoxin (Trx) domains and an Erv-fold sulfhydryl oxidase module. QSOX1 contains two CXXC motifs as redox-active sites that cooperate to relay electrons from reduced thiols of substrate proteins to molecular oxygen. Unlike other disulfide catalysts, QSOX1 is localized downstream of the endoplasmic reticulum. It is found in the Golgi apparatus and secreted from quiescent fibroblasts into the ECM, where it affects ECM composition and especially laminin incorporation. Specifically, QSOX1 affects the incorporation of laminin isoforms that contain an α4 chain (Ilani et al., 2013 ibid), a known marker for tumor progression. Together with the overproduction of QSOX1 in various adenocarcinomas (Antwi et al., Analysis of the Plasma Peptidome from Pancreas Cancer Patients Connects a Peptide in Plasma to Overexpression of the Parent Protein in Tumors, J. Proteome Res. 2009, 8, 10, 4722-4731; Soloviev et al., Elevated Transcription of the Gene QSOX1 Encoding Quiescin Q6 Sulfhydryl Oxidase 1 in Breast Cancer. PLoS ONE 2013, 8(2): e57327) and associated stroma (Finak et al., Stromal gene expression predicts clinical outcome in breast cancer. Nat Med. 2008 May; 14(5):518-27), these findings suggest a possible role of QSOX1 in stimulating tumor cell migration via laminin incorporation and that targeting sites on the protein by interaction with an antibody may accomplish inhibition. An inhibitory monoclonal antibody, which blocks substrate access to the Trx CXXC redox-active site of human QSOX1 has been developed (Grossman et al., 2013 ibid). The mouse anti human antibody, MAb492.1 (also denoted 492), efficiently inhibited human QSOX1 activity, and consequently inhibited adhesion and migration of cancer cells to and through fibroblasts from corresponding tissues, and therefore proposed as an anti-metastatic drug in antibody-based cancer therapy.

### The CUMAB Method of Antibody Humanization Based on Structural and Energy-Based Ranking

The Computational hUMan AntiBody (CUMAB) design method is used for generating stable and producible humanized antibodies that recognize and inhibit QSOX1, based upon the surprising precision of energy-based humanization. CUMAB is highly advantageous to antibody humanization due to its completely automated pipeline, enabling the processing of tens of thousands of grafted constructs in an efficient and time conserving manner, facilitating rapid transition from computational modeling to proof-of-concept experiments to therapeutic applications. The CUMAB method comprises the following steps:

Building a Database of Antibody Germline Sequences: A database of antibody germline sequences was afforded by retrieving antibody germline sequences from the IMGT reference database. For each gene, only the first allele that was annotated as functional was taken. Additionally, genes had to be annotated as not partial and not reverse complementary. If allele one contains more than two cysteines, a different allele was taken that has two cysteines if possible. This filtering scheme resulted in 54 heavy chain V gene sequences, 6 heavy chain J gene sequences, 39 light chain kappa V gene sequences, 5 light chain kappa J gene sequences, 30 light chain lambda V gene sequences, and 5 light chain lambda J gene sequences.

Thereafter an all-versus-all recombination of these four segments: V and J for both light and heavy chains, and for kappa and lambda light chains, was performed separately, resulting in 63,180 sequences for kappa light chains and 48,600 for lambda light chains.

Computational CDR grafting: Starting from a published atomic structure (source from the PDB) of the parental antibody, HMMer (Wheeler et al, nhrnmer: DNA homology search with profile HMMs, Bioinformatics, Volume 29, Issue 19, 1 Oct. 2013, Pages 2487-2489) was used to identify the segments of the sequence corresponding to the variable region and classify the light chain as kappa or lambda. For each germline sequence corresponding to the light chain classification, the CDRs of the germline sequence are replaced with the CDRs of the parental antibody. Any sequence that contains an Asn-Gly or Asn-X-Ser/Thr (where X is not pro) outside of the CDRs was removed, resulting in more than 20,000 unique sequences per one parental antibody.

Energy ranking of CDR-grafted sequences: In cases where a crystal structure is provided, as a first step, the crystal structure of the parental antibody is relaxed through cycles of sidechain and harmonically constrained backbone minimization and combinatorial sidechain packing in the entire Fv. In cases where a bound structure is provided, residues in the interface between the Fv and the antigen were identified using Rosetta and held fixed during the relaxation. If the structure was given in a complex with the antigen, the entire antibody Fv-antigen complex was relaxed.

Each CDR-grafted germline sequence was then threaded onto the relaxed Fv structure and relaxed in the same manner with fewer cycles. Sequences were ranked according to all atom energy using the ref2015 score function. Any model that has a Ca-carbonyl O RMSD of greater than or equal to 0.5 Å in any of the CDRs was excluded from further consideration.

Sequences were clustered according to V gene subgroup as defined by IMGT, meaning that only one sequence was taken from each V gene combination. Sequences were visually inspected and, in some cases, the highest ranking representative for a cluster was replaced with a slightly lower ranking one in order to re-use sequences in different clusters and thus minimize cloning.

When starting from a calculated (in silico) model rather than experimental structure, the pipeline was almost identical, with the only difference being that sequences were not excluded for deviating from the model structure due to uncertainties in modeling.

Computational specificity determining residue (SDR) grafting: The parental antibody was classified as having a kappa or lambda light chain as described above. Rosetta was used to identify residues in the interface between the antibody Fv and the antigen. Antibody germline sequences were selected using the following criteria: the sequences must have the same CDR length in all CDRs excluding H3. Additionally, the sequences must have an H3 length that is equal to or shorter than the H3 length compared to the parental antibody. If the H3 length is shorter than the parental antibody, a number of residues equal to the difference in length of the two H3s from the parental antibody are inserted into the germline sequence. The germline sequences are then threaded and relaxed as described above. Sequences were clustered according to V gene subgroup and heavy J gene subgroup.

According to some embodiments, the computational method used to design or identify the humanized antibodies of the present invention comprises the steps of:
i) providing a structural model of a non-human antibody having an affinity to human QSOX1 (a parental Ab) and identifying amino acid residues of at least one complementarity-determining region (CDR) in the structural model;
ii) generating all combinations of antibody segments derived from a plurality of human antibody germline sequences, and replacing corresponding amino acid residues in each of the combinations with the amino acid residues of the CDR, to thereby creating a library of grafted human antibody sequences;
iii) threading each of the grafted human antibody sequences on the structural model to thereby obtain a plurality of threaded grafted human antibody structures, and subjecting each of the threaded grafted human antibody structures to constrained energy minimization (constrained structural relaxation) to thereby obtain a plurality of relaxed grafted human antibody structures;
iv) ranking the plurality of relaxed grafted human antibody structures by an energy score (low-energy designs are preferred);
v) clustering the plurality of relaxed grafted human antibody structures according to V/J gene families to thereby obtain an energy ranked and gene family clustered library of humanized antibody designs; and
vi) expressing at least one humanized antibody design from at least one cluster of humanized antibody designs and selecting at least one humanized antibody design having an affinity to the antigen of interest, thereby obtaining the humanized antibody having an affinity to the antigen of interest.

In some embodiments, the antibody segments are selected from the group consisting of heavy chain variable (V) gene segment, light chain variable (V) gene segment, heavy chain joining (J) gene segment, light chain joining (J) gene segment, kappa gene segment, and lambda gene segment.

The term “antigen” as used herein refers to a molecule or a portion of a molecule capable of eliciting antibody formation and for being specifically bound by an antibody. An antigen may have one or more than one epitope. The specific binding referred to above is meant to indicate that the antigen will react, in a highly selective manner, with its corresponding antibody and not with the multitude of other antibodies which may be evoked by other antigens. An antigen according to some embodiments of the present invention is a human QSOX1 protein or an immunogenic portion thereof.

A binding affinity or an inhibition constant can be quantified using known methods such as, Surface Plasmon Resonance (SPR) (described, for example in Scarano S, Mascini M, Turner A P, Minunni M. Surface plasmon resonance imaging for affinity-based biosensors. Biosens Bioelectron. 2010, 25: 957-66), and can be calculated using, e.g., a dissociation constant, Kd or an inhibition constant (Ki), such that a lower Kd and Ki values reflect higher affinity and inhibition.

According to one aspect, the present invention provides a humanized antibody or an antigen-binding fragment thereof comprising at least the antigen binding portion, which specifically binds to human QSOX1, said antibody or antigen-binding fragment thereof have a Ki of 5×10⁻⁹M or lower to human QSOX1.

In certain embodiments, the Ki of a humanized antibodies or antigen binding fragment thereof for binding human QSOX1 is less than about 5 nM, 4 nM, 3 nM, 2 nM, 1 nM, 0.5 nM, 0.1 nM, 0.05 nM, 0.01 nM, or 0.001 nM.

Half maximal effective concentration (EC50) refers to the concentration of the antibody that induces a response halfway between the baseline and maximum after a specified exposure time.

Antibodies, or immunoglobulins, comprise two heavy chains linked together by disulfide bonds and two light chains, each light chain being linked to a respective heavy chain by disulfide bonds in a “Y” shaped configuration. Proteolytic digestion of an antibody yields Fv (Fragment variable) and Fc (Fragment crystallizable) domains. The antigen binding domains, Fab, include regions where the polypeptide sequence varies. The term F(ab′)₂ represents two Fab′ arms linked together by disulfide bonds. Each heavy chain has at one end a variable domain (V_{H}) followed by a number of constant domains (C_{H}). Each light chain has a variable domain (V_{L}) at one end and a constant domain (C_{L}) at its other end, the light chain variable domain being aligned with the variable domain of the heavy chain and the light chain constant domain being aligned with the first constant domain of the heavy chain (CH1). The variable domains of each pair of light and heavy chains form the antigen-binding site. The variable domains on the light and heavy chains have the same general structure and each domain comprises four framework regions, whose sequences are relatively conserved, joined by three hyper-variable domains known as complementarity determining regions (CDRs). These domains contribute specificity and affinity of the antibody to its target antigen.

There are several methods known in the art for determining the CDR sequences of a given antibody molecule, but there is no standard unequivocal method. Determination of CDR sequences from antibody heavy and light chain variable regions can be made according to any method known in the art, including, but not limited to, the methods known as Kabat, Chothia, and IMGT detailed below.

In certain embodiments the CDRs of the antibodies described herein can be defined by a method selected from Kabat, Chothia, IMGT, Aho, AbM, or combinations thereof. A selected set of CDRs according to the present invention may include sequences identified by more than one method, namely, some CDR sequences may be determined using Kabat and some using IMGT, for example. According to some embodiments, the CDR sequences of the antibody variable regions are determined using the Kabat and/or Chothia methods. According to some specific embodiments, CDR determination is according to Kabat.

“Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (Wu T. T and Kabat E. A., J Exp Med, 1970; 132:211-50), and “Sequences of Proteins of Immunological Interest,” 5th Ed. Public Health Service, 1991, National Institutes of Health, Bethesda, M D (“Kabat” numbering scheme), Al-Lazikani et al., (1997) JMB 273,927-948 (“Chothia” numbering scheme); MacCallum et al., J. Mol. Biol. 1996, 262:732-745, “Antibody-antigen interactions: Contact analysis and binding site topography,” J. Mol. Biol. 262, 732-745.” (“Contact” numbering scheme); Lefranc MP et al., “IMGT unique numbering for immunoglobulin and T cell receptor variable domains and Ig superfamily V-like domains,” Dev Comp Immunol, 2003 January;27(1):55-77 (“IMGT” numbering scheme); Honegger A and Pluckthun A, “Yet another numbering scheme for immunoglobulin variable domains: an automatic modeling and analysis tool,” J Mol Biol, 2001, June 8;309(3):657-70, (“Aho” numbering scheme); and Whitelegg NR and Rees A R, “WAM: an improved algorithm for modelling antibodies on the WEB,” Protein Eng. 2000, December;13(12):819-24 (“AbM” numbering scheme.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Alterations (e.g., substitutions) may be made in CDRs, e.g., to improve antibody affinity. Such alterations may be made in CDR encoding codons with a high mutation rate during somatic maturation (See e.g., Chowdhury, Methods Mol. Biol. 207:179-196 (2008)), and the resulting variant can be tested for binding affinity. Affinity maturation (e.g., using error-prone PCR, chain shuffling, randomization of CDRs, or oligonucleotide-directed mutagenesis) can be used to improve antibody affinity (See e.g., Hoogenboom et al. in Methods in Molecular Biology 178:1-37 (2001)). CDR residues involved in antigen binding may be specifically identified, e.g., using alanine scanning mutagenesis or modeling (See e.g., Cunningham and Wells Science, 244:1081-1085 (1989)). CDR-H3 and CDR-L3 in particular are often targeted.

When the term “CDR having a sequence”, or a similar term is used, it includes options wherein the CDR comprises the specified sequences and also options wherein the CDR consists of the specified sequence.

The antigen specificity of an antibody is based on the hyper variable region (HVR), namely the six unique CDR sequences of both light and heavy chains that together form the antigen-binding site.

According to an aspect, the present invention provides an antibody or antigen-binding fragment thereof comprising a set of six CDR sequences wherein, heavy chain CDR1 is VSGFSLTGYGVN (SEQ ID NO: 3); heavy chain CDR2 is WLGMIWGDGRTD (SEQ ID NO: 4); heavy chain CDR3 is ASDYYGSGSFAY (SEQ ID NO: 5); light chain CDR1 is KASQDVSTAVA (SEQ ID NO: 6); light chain CDR2 is LLIHSASYRY (SEQ ID NO: 7); and light chain CDR3 is QQHYSIPLT (SEQ ID NO: 8).

The isotype of the heavy chain (gamma, alpha, delta, epsilon or mu) determines immunoglobulin class (IgG, IgA, IgD, IgE or IgM, respectively). The light chain is either of two isotypes (kappa, x or lambda, X). Both isotopes are found in all antibody classes. The humanized antibodies of the present invention are typically IgG antibodies. Human IgG antibodies consists of four subclasses (IgG1, IgG2, IgG3 and IgG4) each containing a different heavy chain. They are highly homologous and differ mainly in the hinge region and the extent to which they activate the host immune system. IgG1 and IgG4 contain two inter-chain disulfide bonds in the hinge region, IgG2 has 4 and IgG3 has 11. The antibodies of the present invention may contain any human IgG subtype. According to some embodiments, the humanized antibodies of the present invention comprise a human IgG1 constant region.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. According to some embodiments, some FR residues in a humanized antibody may be substituted with corresponding residues from a non-human antibody (e.g., the antibody from which the CDR residues are derived), e.g., to restore or improve antibody specificity or affinity.

Humanized antibodies according to the invention includes intact antibodies, as well as fragments thereof, including proteolytic fragments such as Fab or F(ab′)₂ fragments and single chain antibodies (e.g. scFv).

The terms “antibody having the antigen-binding portion/domain of an antibody” and “antigen-binding fragment” as used herein are intended to include not only intact immunoglobulin molecules of any isotype and generated by any cell line or microorganism, but also the antigen-binding reactive fraction thereof, including, but not limited to, the Fab fragment, the Fab′ fragment, the F(ab′)₂ fragment, the variable portion of the heavy and/or light chains thereof, Fab mini-antibodies (e.g., WO 93/15210, U.S. patent application Ser. No. 08/256,790, WO 96/13583, U.S. patent application Ser. No. 08/817,788, WO 96/37621, U.S. patent application Ser. No. 08/999,554), and single-chain antibodies incorporating such reactive fraction, as well as any other type of molecule in which such antibody reactive fraction has been physically inserted. Such molecules may be provided by any known technique, including, but not limited to, enzymatic cleavage, peptide synthesis or recombinant techniques.

An “antibody fragment” according to the present invention is an “antigen-binding fragment” that comprise only a portion of an intact antibody, including the antigen binding portion (or domain) of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 1989, 341, 544-546) which consists of a VH domain; (vii) isolated CDR regions; (viii) F(ab′)₂ fragments, a bivalent fragment including two Fab′ fragments linked by a disulfide bridge at the hinge region; (ix) single chain antibody molecules (e.g. single chain Fv; scFv) (Bird et al., Science 1988, 242, 423-426; and Huston et al., Proc. Natl. Acad. Sci. (USA) 1988, 85,5879-5883); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 1993, 90, 6444-6448); (xi) “linear antibodies” comprising a pair of tandem Fd segments (VH-CH1-VH-CH1) which, together with complementary light chain polypeptides, form a pair of antigen binding regions (Zapata et al. Protein Eng., 1995, 8, 1057-1062; and U.S. Pat. No. 5,641,870).

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can now be produced directly by recombinant host cells. For example, the antibody fragments can be isolated from antibody phage libraries. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv).

Single chain antibodies can be single chain composite polypeptides having antigen binding capabilities and comprising amino acid sequences homologous or analogous to the variable regions of an immunoglobulin light and heavy chain i.e., linked V_{H}-V_{L} or single chain Fv (scFv). Techniques for the production of single-chain antibodies (for example, U.S. Pat. No. 4,946,778) can be adapted to produce single-chain fragments from the humanized antibodies of the present invention.

Sequence identity is the percentage of amino acids or nucleotides which match exactly between two different sequences. Sequence similarity permits conservative substitution of amino acids to be determined as identical amino acids. The polynucleotide sequences described herein may be codon-optimized for expression in specific cells, such as human cells. Codon optimization does not change the encoded amino acid sequences of the antibody's chain but may, for example, increase the expression in cells.

Analogs and derivatives of the humanized antibodies and the antigen-binding fragments described in the present invention, are also within its scope as long as they retain the activity, stability and producibility properties of the parent antibody.

Analogs and derivatives of the antibody sequences are also within the scope of the present application. These include, but are not limited to, conservative and non-conservative substitution, insertion, and deletion of amino acids within the sequence. Such modification and the resultant antibody analog or variant are within the scope of the present invention as long as they confer, or even improve the binding of the humanized antibody to the human QSOX1.

According to some embodiments, an analog or a derivative of a humanized antibody or antibody fragment has at least 90% sequence identity with any of the chains of the reference antibody sequence. According to certain embodiments, the analog or derivative of the humanized antibody or antigen-binding fragment thereof has at least 91, 92, 93, 94, 95, 96, 97, 98 or 99% sequence identity with a variable region of the reference antibody sequence. Each possibility represents a separate embodiment of the invention.

According to some embodiments, the analog or derivative has at least 95, 96, 97, 98 or 99% sequence similarity or identity with an antibody light or heavy chain variable regions or with the light or heavy chains described above. According to some embodiments, the analog comprises no more than one amino acid substitution, deletion, or addition to one or more CDR sequences of the hypervariable region, namely, any one of the CDR sequences set forth in SEQ ID NOs: 3-8. Each possibility represents a separate embodiment of the present invention. According to some embodiments, the amino acid substitution is a conservative substitution.

According to some embodiments, the antibody or antigen-binding fragment comprises a hypervariable region (HVR) having light and heavy chain CDR sequences defined above, in which 1, 2, 3, 4, or 5 amino acids were substituted, deleted and/or added. According to some embodiments, the humanized antibody or antigen-binding fragment comprises a HVR having light and heavy chain CDR sequences defined above, in which one amino acid was substituted. According to specific embodiments, the antibody or antigen-binding fragment comprises a CDR as defined above, in which one amino acid was substituted. Each possibility represents a separate embodiment of the invention.

Variants and analogs according to the invention also may be made that conserve the overall molecular structure of the encoded proteins. Given the properties of the individual amino acids, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved.

Conservative substitutions of amino acids are known to those skilled in the art and include replacement of one amino acid with another having the same type of functional group or side chain, e.g., aliphatic, aromatic, positively charged, negatively charged. These substitutions may enhance oral bioavailability, penetration, and targeting to specific cell populations, immunogenicity, and the like. One of skill will recognize that individual substitutions, deletions or additions to a peptide, polypeptide, or protein sequence which alters, adds, or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. For example, according to one table known in the art, the following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

The term “antibody conjugate” as used herein refers to any molecule comprising the humanized antibody or antigen-binding fragment of the present invention. For example, fusion proteins in which the humanized antibody or an antigen-binding-fragment thereof is linked to another entity, such as an anti-cancer drug or an identifiable moiety, is considered an antibody conjugate.

The humanized antibodies described herein are encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors”. Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes or can be artificial. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a chromosomal location. Vectors can comprise sequences that direct site-specific integration into a defined location or restricted set of sites in the genome (e.g., AttP-AttB recombination). Additionally, vectors can comprise sequences derived from transposable elements.

The nucleic acids encoding the humanized antibodies described herein can be used to infect, transfect, transform, or otherwise render a suitable cell transgenic for the nucleic acid, thus enabling the production of antibodies for commercial or therapeutic uses. Standard cell lines and methods for the production of antibodies from a large-scale cell culture are known in the art. In certain embodiments, the cell is a eukaryotic cell. In certain embodiments, the eukaryotic cell is a mammalian cell. In certain embodiments, the mammalian cell is a cell line useful for producing antibodies is a Chines Hamster Ovary cell (CHO) cell, an NS0 murine myeloma cell, or a PER.C6® cell. In certain embodiments, the nucleic acid encoding the humanized antibody is integrated into a genomic locus of a cell useful for producing antibodies. In certain embodiments, described herein is a method of making a humanized antibody comprising culturing a cell comprising a nucleic acid encoding the humanized antibody under conditions in vitro sufficient to allow production and secretion of said antibody.

Any method known in the art for expressing and purifying antibodies may be used to produce the humanized antibodies of the present invention, including but not limited to the method described in Vazquez-Lombardi et al., Nature Protocols, 2018, 13,1, 99-117.

It should be emphasized that different sequencing methods employed on the same protein or nucleotide sequence may result in slightly different sequences due to technical issues and different primers, particularly in the sequence terminals.

Also included within the scope of the present invention are methods of making the humanized antibodies and fragments specific to human QSOX1. Such methods comprise incubating a cell or cell-line comprising a nucleic acid encoding the humanized antibody in a cell culture medium under conditions sufficient to allow for expression and secretion of the antibody, and further harvesting the antibody from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

### Pharmacology and Methods of Treatments

In pharmaceutical and medicament formulations, the active agent is preferably utilized together with one or more pharmaceutically acceptable carrier(s) and optionally any other therapeutic ingredients. The carrier(s) must be pharmaceutically acceptable in the sense of being compatible with the other ingredients of the formulation and not unduly deleterious to the recipient thereof. The active agent is provided in an amount effective to achieve the desired pharmacological effect, as described above, and in a quantity appropriate to achieve the desired exposure.

The humanized antibodies of the present invention as active ingredients are dissolved, dispersed or admixed in an excipient that is pharmaceutically acceptable and compatible with the active ingredient as is well known. Suitable excipients are, for example, water, saline, phosphate buffered saline (PBS), dextrose, glycerol, ethanol, or the like and combinations thereof. Other suitable carriers are well known to those skilled in the art. In addition, if desired, the composition can contain minor amounts of auxiliary substances such as wetting or emulsifying agents, pH buffering agents.

According to some embodiments, the pharmaceutical composition comprises 1-50 mg/ml of humanized antibody. According to some embodiments, the pharmaceutical composition comprises a basic amino acid. According to some embodiments, the pharmaceutical composition comprises a sugar. According to some embodiments, the pharmaceutical composition comprises a surfactant. According to some embodiments, the pharmaceutical composition comprises a basic amino acid, a sugar and a surfactant. According to some embodiments, the pharmaceutical composition comprises (i) 1-10 mg/ml of basic amino acid; (ii) 10-200 mg/ml of a sugar; (iii) 0.01-1 mg/ml of a surfactant; (iv) 1-50 mg/ml of humanized antibody.

According to some embodiments, the basic amino acid is selected from the group consisting of: Histidine, Arginine, Lysine and Ornithine. Each possibility represents a separate embodiment of the present invention.

The term “sugar” refers to monosaccharides, disaccharides, and polysaccharides, Examples of sugars include, but are not limited to, sucrose, trehalose, dextrose, and others. According to some embodiments, the sugar is selected from the group consisting of: sucrose, trehalose, glucose, dextrose and maltose. Each possibility represents a separate embodiment of the present invention. According to some embodiments, the composition comprises 10-200, 10-100, 50-150 or 70-100 mg/ml of sugar. Each possibility represents a separate embodiment of the present invention.

According to yet other embodiments, the composition comprises polyol, including but not limited to mannitol and sorbitol.

According to some embodiments, the surfactant is a non-anionic. According to some embodiments, the surfactant selected from the group consisting of: polysorbates, sorbitan esters and poloxamers. Each possibility represents a separate embodiment of the present invention. According to some embodiments, the composition comprises 0.01-10, 0.01-1, 0.05-5 or 0.1-1 mg/ml of surfactant. Each possibility represents a separate embodiment of the present invention.

Typically, the humanized antibodies and antigen-binding fragments and conjugates thereof of the present invention will be suspended in a sterile saline solution for therapeutic uses. The pharmaceutical compositions may alternatively be formulated to control release of active ingredient or to prolong its presence in a patient's system. Numerous suitable drug delivery systems are known and include, e.g., implantable drug release systems, hydrogels, hydroxymethylcellulose, microcapsules, liposomes, microemulsions, microspheres, and the like. Controlled release preparations can be prepared through the use of polymers to complex or adsorb the molecule according to the present invention. For example, biocompatible polymers include matrices of poly(ethylene-co-vinyl acetate) and matrices of a polyanhydride copolymer of a stearic acid dimer and sebaric acid. The rate of release of the molecule according to the present invention, i.e., of a humanized antibody or antibody fragment, from such a matrix depends upon the molecular weight of the molecule, the amount of the molecule within the matrix, and the size of dispersed particles.

The pharmaceutical compositions of this invention are formulated for administration by any suitable means, such as intravenously (i.v.), subcutaneously (s.c.), intramuscularly (i.m.), orally, topically, intranasally, intra-arterially, intraarticulary, intralesionally, intratumorally or parenterally. Ordinarily, intravenous (i.v.) administration is used for delivering antibodies. In some embodiments, the humanized antibodies or antibody fragments are administered by infusion.

According to an aspect, the present invention provides a method of treating cancer comprising administering to a subject in need thereof, a pharmaceutical composition comprising a therapeutically effective amount of a humanized antibody or antibody fragment or conjugate thereof described herein.

As used herein the term “subject”, “individual”, or “patient” refers to individuals diagnosed with, suspected of being afflicted with, or at-risk of developing at least one disease for which the described compositions and method are useful for treating. According to some embodiments the individual is a mammal. According to some embodiments, the individual is a human.

It will be apparent to those of ordinary skill in the art that the therapeutically effective amount of an antibody molecule according to the present invention will depend, inter alia upon the administration schedule, the unit dose of molecule administered, whether the molecule is administered in combination with other therapeutic agents, the immune status and health of the patient, the therapeutic activity of the molecule administered, its persistence in the blood circulation, and the judgment of the treating physician.

As used herein the term “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The term “cancer” refers to or describes the physiological condition in mammals that is typically characterized by unregulated cell growth. The cancer amendable for treatment by the antibody molecules of the present invention includes, but is not limited to: carcinoma, lymphoma, blastoma, sarcoma, and leukemia or lymphoid malignancies. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high-grade immunoblastic NHL; high-grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. According to some embodiments, the cancer is a solid cancer or tumor. According to some embodiments, the cancer is selected from the group consisting of: a prostate cancer, a lung cancer, a breast cancer, a cervical cancer, an urachus cancer, a vaginal cancer, a colon cancer, an esophagus cancer, a pancreatic cancer, a throat cancer, a stomach cancer and a myeloid leukemia. The cancerous conditions amendable for treatment of the invention include metastatic cancers.

The pharmaceutical composition according to the present invention may be administered together or in combination with an anti-cancer composition.

As used herein the term “combination” or “combination treatment” can refer either to concurrent administration of the articles to be combined or sequential administration of the articles to be combined. As described herein, when the combination refers to sequential administration of the articles, the articles can be administered in any temporal order.

The term “treatment” as used herein refers to both therapeutic treatment and prophylactic or preventative measures. Those in need of treatment include those already with the disorder as well as those in which the disorder is to be prevented.

According to some embodiments, the method of treating cancer comprises administering the pharmaceutical composition as part of a treatment regimen comprising administration of at least one additional anti-cancer agent or treatment.

According to some embodiments, the additional anti-cancer agent is selected from the group consisting of an antimetabolite, a mitotic inhibitor, a taxane, a topoisomerase inhibitor, a topoisomerase II inhibitor, an asparaginase, an alkylating agent, an antitumor antibiotic, an immune-modulator, a checkpoint inhibitor, an antibody targeting a tumor antigen, and combinations thereof. Each possibility represents a separate embodiment of the invention.

According to some embodiments, the antimetabolite is selected from the group consisting of cytarabine, fludarabine, fluorouracil, mercaptopurine, methotrexate, thioguanine, gemcitabine, and hydroxyurea. According to some embodiments, the mitotic inhibitor is selected from the group consisting of vincristine, vinblastine, and vinorelbine. According to some embodiments, the topoisomerase inhibitor is selected from the group consisting of topotecan and irinotecan. According to some embodiments, the alkylating agent is selected from the group consisting of busulfan, carmustine, lomustine, chlorambucil, cyclophosphamide, cisplatin, carboplatin, ifosfamide, mechlorethamine, melphalan, thiotepa, dacarbazine, and procarbazine. According to some embodiments, the antitumor antibiotic is selected from the group consisting of bleomycin, dactinomycin, daunorubicin, doxorubicin, idarubicin, mitomycin, mitoxantrone, and plicamycin. According to some embodiments, the topoisomerase II is selected from the group consisting of etoposide and teniposide. Each possibility represents a separate embodiment of the present invention.

According to some particular embodiments, the additional anti-cancer agent is selected from the group consisting of bevacizumab, carboplatin, cyclophosphamide, doxorubicin hydrochloride, gemcitabine hydrochloride, topotecan hydrochloride, thiotepa, and combinations thereof. Each possibility represents a separate embodiment of the present invention.

Humanized antibodies according to the present invention may be also used as part of combined therapy with at least immuno-modulator, an activated lymphocyte cell, a kinase inhibitor or a chemotherapeutic agent.

According to some embodiments, the anti-cancer agent is an immuno-modulator, whether agonist or antagonist, such as antibody against an immune checkpoint molecule.

Checkpoint immunotherapy blockade has proven to be an exciting new venue of cancer treatment. Immune checkpoint pathways consist of a range of co-stimulatory and inhibitory molecules which work in concert in order to maintain self-tolerance and protect tissues from damage by the immune system under physiological conditions. Tumors take advantage of certain checkpoint pathways in order to evade the immune system. Therefore, the inhibition of such pathways has emerged as a promising anti-cancer treatment strategy.

The anti-cytotoxic T lymphocyte 4 (CTLA-4) antibody ipilimumab (approved in 2011) was the first immunotherapeutic agent that showed a benefit for the treatment of cancer patients. The antibody interferes with inhibitory signals during antigen presentation to T cells. Anti-programmed cell death 1 (PD-1) antibody pembrolizumab (approved in 2014) blocks negative immune regulatory signaling of the PD-1 receptor expressed by T cells. An additional anti-PD-1 agent was filed for regulatory approval in 2014 for the treatment of non-small cell lung cancer (NSCLC). Active research is currently exploring many other immune checkpoints, among them: CEACAM1, NKG2A, B7-H3, B7-H4, VISTA, lymphocyte activation gene 3 (LAG3), CD137, OX40 (also referred to as CD134), and killer cell immunoglobulin-like receptors (KIR).

According to other embodiments the additional anti-cancer agent is a chemotherapeutic agent. A chemotherapy agent, which could be administered together with the antibody according to the present invention, or separately, may comprise any such agent known in the art exhibiting anti-cancer activity, including but not limited to: mitoxantrone, topoisomerase inhibitors, spindle poison from vinca: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabine; podophyllotoxins: etoposide, irinotecan, topotecan, dacarbazine; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, and megestrol acetate.

According to some embodiments, the chemotherapeutic agent is selected from alkylating agents, antimetabolites, folic acid analogs, pyrimidine analogs, purine analogs and related inhibitors, vinca alkaloids, epipodophyllotoxins, antibiotics, L-asparaginase, topoisomerase inhibitor, interferons, platinum coordination complexes, anthracenedione substituted urea, methyl hydrazine derivatives, adrenocortical suppressant, adrenocorticosteroids, progestins, estrogens, antiestrogen, androgens, antiandrogen, and gonadotropin-releasing hormone analog.

According to another embodiment, the chemotherapeutic agent is selected from the group consisting of 5-fluorouracil (5-FU), leucovorin (LV), irinotecan, oxaliplatin, capecitabine, paclitaxel and docetaxel. One or more chemotherapeutic agents can be used.

According to still another aspect the present invention provides a method of treating cancer in a subject in need thereof comprising administering to said subject a therapeutically effective amount of a humanized antibody or antigen-binding fragment or conjugate according to the present invention.

Toxicity and therapeutic efficacy of the compositions described herein can be determined by standard pharmaceutical procedures in cell cultures or experimental animals, e.g., by determining the IC50 (the concentration which provides 50% inhibition) and the maximal tolerated dose for a subject compound. The data obtained from these cell culture assays, and animal studies can be used in formulating a range of dosages for use in humans. The dosage may vary depending inter alia upon the dosage form employed, the dosing regimen chosen, the composition of the agents used for the treatment and the route of administration utilized, among other relevant factors. The exact formulation, route of administration and dosage can be chosen by the individual physician in view of the patient's condition. Depending on the severity and responsiveness of the condition to be treated, dosing can also be a single administration of a slow-release composition, with course of treatment lasting from several days to several weeks or until cure is affected or diminution of the disease state is achieved. The amount of a composition to be administered will, of course, be dependent on the subject being treated, the severity of the affliction, the manner of administration, the judgment of the prescribing physician, and all other relevant factors.

The term “administering” or “administration of” a substance, a compound or an agent to a subject can be carried out using one of a variety of methods known to those skilled in the art. For example, a compound or an agent can be administered enterally or parenterally. Enterally refers to administration via the gastrointestinal tract including per os, sublingually or rectally. Parenteral administration includes administration intravenously, intradermally, intramuscularly, intraperitoneally, subcutaneously, ocularly, sublingually, intranasally, by inhalation, intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

Antibodies are generally administered in the range of about 0.1 to about 20 mg/kg of patient weight, commonly about 0.5 to about 10 mg/kg, and often about 1 to about 5 mg/kg. lower or higher doses are also possible if required and if tolerated. In this regard, it is sometimes favored to use antibodies having a circulating half-life of at least 12 hours, preferably at least 4 days, more preferably up to 21 days. In some cases, it may be advantageous to administer a large loading dose followed by periodic (e.g., weekly) maintenance doses over the treatment period. Antibodies can also be delivered by slow-release delivery systems, pumps, and other known delivery systems for continuous infusion.

The present invention further comprises, according to another aspect, a method of determining or quantifying human QSOX1 in a sample, the method comprising contacting a biological sample with an antibody or with an antigen-binding fragment according to the invention and measuring the level of complex formation.

The present invention further discloses methods for diagnosis, prognosis and theragnosis of cancer. These methods include but are not limited to, staging and grading the disease, determining cancer recurrence, determining disease aggressiveness, determining patient prognosis following treatment, and providing information for assisting decisions regarding initial or further treatment with the humanized antibodies of the present invention.

According to an aspect, the present invention provides a diagnostic, prognostic and/or theragnosis method of cancer in a subject, the method comprises the step of determining the expression level of human QSOX1 in a biological sample of said subject using at least one humanized antibody as described herein.

The term “biological sample” encompasses a variety of sample types obtained from an organism that may be used in a diagnostic or monitoring assay. The term encompasses blood and other liquid samples of biological origin, solid tissue samples, such as a biopsy specimen, or tissue cultures or cells derived there from and the progeny thereof. Additionally, the term may encompass circulating tumor or other cells. The term specifically encompasses a clinical sample, and further includes cells in cell culture, cell supernatants, cell lysates, serum, plasma, urine, amniotic fluid, biological fluids including aqueous humour and vitreous for eyes samples, and tissue samples. The term also encompasses samples that have been manipulated in any way after procurement, such as treatment with reagents, solubilization, or enrichment for certain components.

Determining the expression level of human QSOX1 can be performed with any of the humanized antibodies, fragments and conjugates described herein, wherein the antibody is labeled with a detectable prob, such as a fluorescent, colorimetric or radioactive prob. Determining the expression can be performed, for example, by ELISA.

The method of the invention can further comprise the step of comparing said level of expression to a control level.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the appended claims.

The following examples are presented in order to more fully illustrate some embodiments of the invention. They should, in no way be construed as limiting the scope of the invention.

### EXAMPLES

Reference is now made to the following examples, which together with the above descriptions, illustrate the invention in a non-limiting fashion.

Generally, the nomenclature used herein and the laboratory procedures utilized in the present invention include computational, molecular, biochemical, microbiological, immunological and recombinant DNA techniques. Such techniques are well known in the art. Other general references referring to well-known procedures are provided throughout this document for the convenience of the reader.

### Example 1. Design of Humanized Antibodies Employing a Computational Method which Conducts Structural and Energy-Based Ranking for Maximal Efficiency

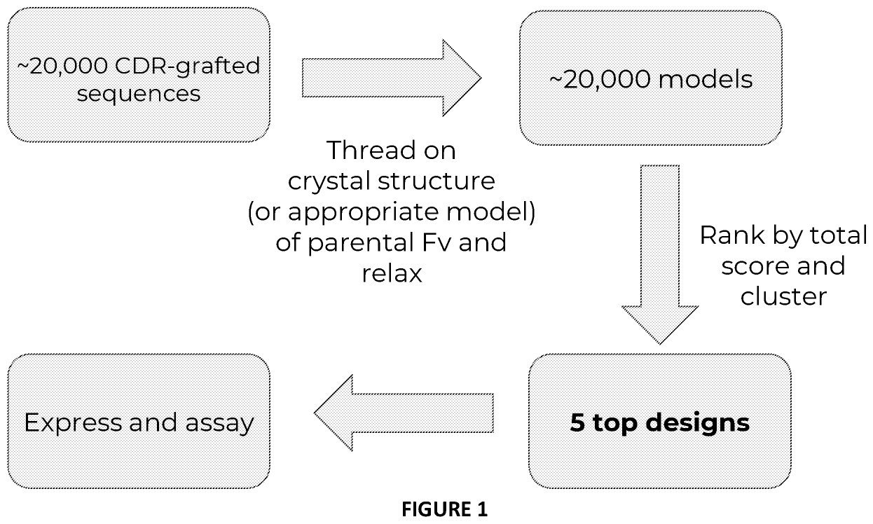

The Computational hUMan AntiBody design method (CUMAB, schematically described in FIG. 1) has been used for generating improved humanized antibodies that recognize and inhibit QSOX1, based on structural and energy-based ranking. This antibody is challenging for humanization since chimerizing its mouse Fv with a human IgG1 constant region leads to a complete loss of expression in HEK293 cells (Warszawski et al., Optinizing antibody affinity and stability by the automated design of the variable light-heavy chain interfaces. PLoS Comput Biol 2020, 16(10): e1008382). This antibody has also undergone previous unsuccessful attempts at humanization using, for example the AbLIFT method, which uses atomistic design calculations to improve the molecular interactions between the Fv light and heavy domains (Warszawski et al., 2020). One of the antibody designs, AbLIFT18, rescued HEK293 expression and QSOX1 inhibition but presented an incompletely humanized antibody. Using a preliminary version of the humanization workflow provided herein, ten designs were tested experimentally, including the lowest-energy five designs after clustering as well as all the combinations of heavy and light chains observed in these five; these combinations were all within the top 2,700 of 26,000+ designs (ranking using updated method). The difference between this preliminary protocol and the current was that the H1 CDR definition in the preliminary version excluded two N-terminal amino acid positions relative to the final protocol. In addition, in this exemplary use of the method provided herein, CDR mutations that were implemented in the control AbLIFT18 antibody design were incorporated relative to the mouse antibody. These ten designs were formatted as IgG1 full-length antibodies and expressed in HEK293 cells, followed by protein G affinity purification. A qualitative dot-blot analysis showed that many antibody designs expressed as well as antibody AbLIFT18 but none showed comparable QSOX1 inhibition levels. Electrophoretic-mobility analysis in denaturing conditions revealed that the designs' apparent molecular mass was heterogeneous and different from that of the parental antibody, suggesting that these designs were aggregated or misfolded. Visual inspection of the design models to find the source of these stability problems revealed that one of the humanization mutations, heavy chain Val24Phe (Kabat numbering), which was present in nearly all the designs, was structurally incompatible with the H1 backbone, suggesting that the preliminary CDR definitions failed to include amino acids that were important for humanization.

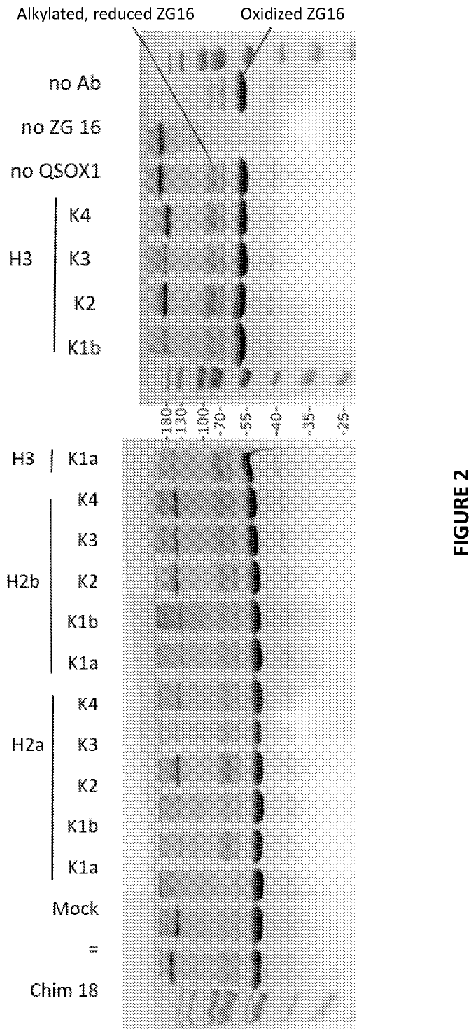

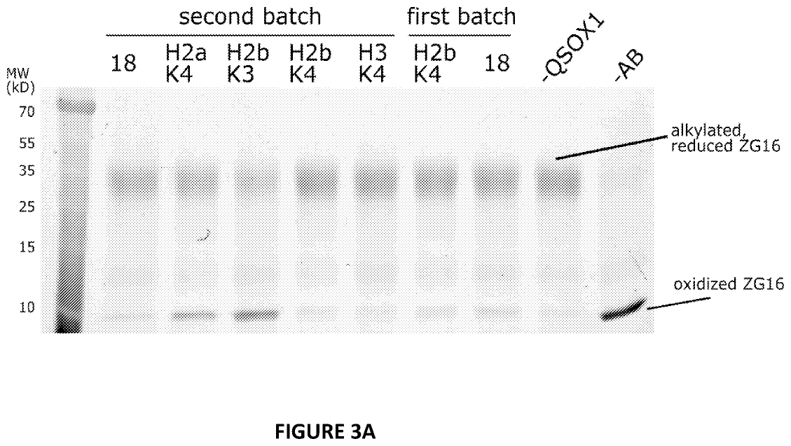

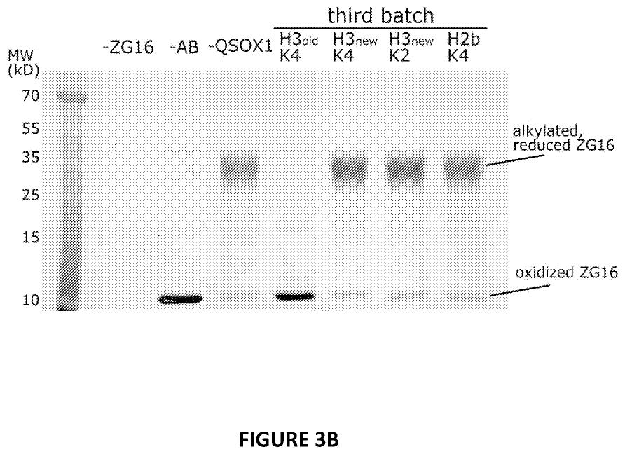

The design calculations were repeated using the CUMAB final version of the CDR definitions which incorporated Kabat positions 24 and 25 in the H1 definition. This method run started directly from the parental mouse antibody without including the mutations in the CDRs from design AbLIFT18. Fifteen designs were experimentally tested (top-five ranked designs and the combinations of light and heavy chains from these five) of which 12 showed comparable expression levels on a dot-blot analysis to AbLIFT18. Unlike the first humanization attempt, and as demonstrated in FIGS. 2, 3A and 3B, electrophoretic analysis using SDS-PAGE revealed that seven designs showed comparable expression levels to the control antibody AbLIFT18 with no significant evidence of misfolding or aggregation. Four variants show high activity: H3K4, H2bK4, H3newK4 and H3newK2. For the electrophoretic analysis, HEK culture supernatants were run on non-reducing SDS-PAGE. The expression is tested qualitatively by intensity of the ˜150 KDa band in each well in comparison to the positive control. To test activity of the activity of the antibodies, zymogen granule membrane protein 16 (ZG16) was used as a QSOX1 substrate. ZG16 contains two cysteines in a flexible, accessible CxxC motif that can be oxidized in vitro by QSOX1. Prior to the assay, ZG16 is reduced by incubation with dithiothreitol (DTT), excess DTT is removed, and reactions of the reduced ZG16 and QSOX1 are initiated in presence of the various antibody variants. The reaction is stopped by adding maleimide-functionalized polyethylene glycol (mal-PEG) of molecular weight 5000 Da, which covalently modifies any residual free thiols, resulting in an SDS-PAGE band shift. Chim18 and 2, used as positive controls, are chimeric versions of the original mouse antibody with additional stabilizing mutations, and were previously shown to have comparable expression levels to the parental antibody.

Remarkably, two of the designed antibodies, H2bK4 and H3K4, showed equivalent levels of QSOX1 inhibition to the parental mouse antibody when added at stoichiometric ratios and one (H3K4) exhibited substantial levels of inhibition even at sub stoichiometric ratios (Example 3). Thus, these two designs recapitulated the parental mouse expression and activity using a humanized Fv framework. These results also emphasize the importance of structurally appropriate CDR definitions for humanization success.

As an additional comparison with conventional humanization strategies, the anti-QSOX1 antibody was subjected to humanization using the “consensus” approach. In this humanization approach, the framework is taken from a sequence-based consensus of V gene subgroups (in this specific case IGKV1 and IGHV4). The designed antibody, however, failed to express and its binding to human QSOX1 was therefore not tested.

Taken together, these results suggest that simply grafting CDRs from an animal antibody onto the closest (or consensus) human germline often fails to recapitulate the animal antibody's stability and functional properties, as has been observed in decades of antibody engineering. By contrast, some of the top-ranked designs from energy-based humanization were well expressed, stable and showed good affinity values to the antigen, and these typically do not derive from homologous subgroups.

### Example 2. Antibody Production and Purification

Antibodies were produced by transient transfection of plasmids (heavy and light chain pairs) into HEK293F cells. Transfection was done using the PEI Max reagent with a 1:3 ratio (w/w) of DNA to PEI at a concentration of 1 million cells per milliliter. Six days after transfection, the culture medium was collected and centrifuged for 15 min at 500 g to pellet cells. The supernatant was then centrifuged for 15 min at 3000 g to pellet any remaining particulate matter. The supernatant from this second centrifugation was filtered through a 0.45 mm filter, and the proteins were purified by Protein G chromatography. Buffer was then exchanged to PBS.

### Example 3. Quantitative QSOX1 Inhibition Assay

A Clark type oxygen electrode was used to monitor changes in dissolved oxygen concentration as a measure of QSOX1 activity. Antibody was mixed with QSOX1, and reactions were initiated by injection of the model substrate dithiothreitol (DTT). QSOX1 and DTT were at fixed concentrations of 25 nM and 200 μM, respectively, and the antibody concentration was varied. The initial slope of dissolved oxygen concentration was recorded for each antibody concentration. Reactions were performed in duplicate, and the results for each antibody concentration were averaged. Relative activity compared to the uninhibited reaction was plotted against antibody concentration and fitted to the Morrison Ki equation for a tight binding competitive inhibitor, to yield the inhibitory constant (Ki):

$$\begin{matrix}\frac{Vi}{Vo} = 1 - \frac{( [ E ] + [ I ] + {K}_{i}^{app} ) - \sqrt{{( [ E ] + [ I ] + {K}_{i}^{app} )}^{2} - 4 [ E ] [ I ]}}{2 [ E ]} \\ {K}_{i}^{app} = {K}_{i} ( 1 + \frac{[ S ]}{{K}_{m}} )\end{matrix}$$

Measurements were performed at 25° C. in 50 mM potassium phosphate buffer, pH 7.5, 65 mM NaCl, and 1 mM ethylenediaminetetraacetic acid (EDTA).

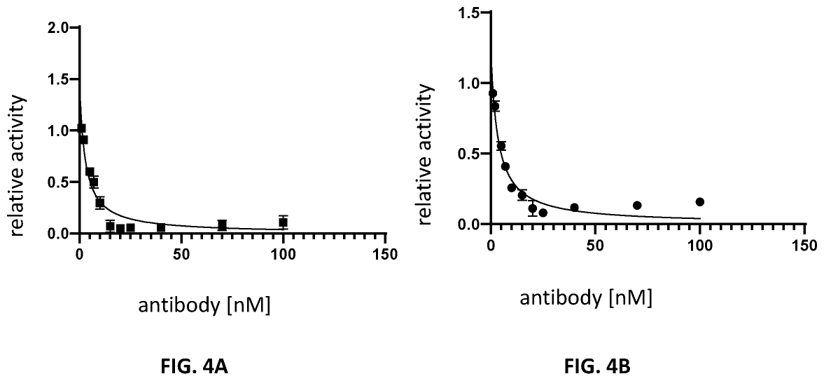

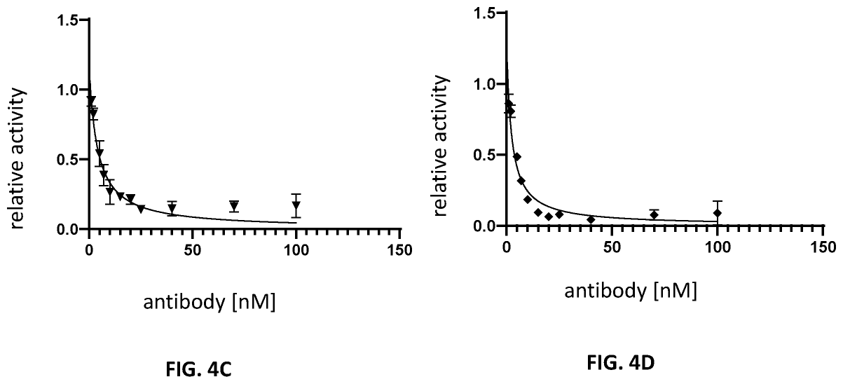

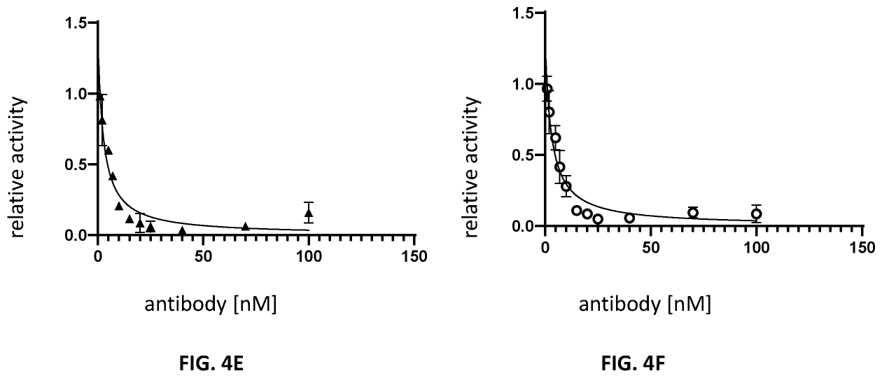

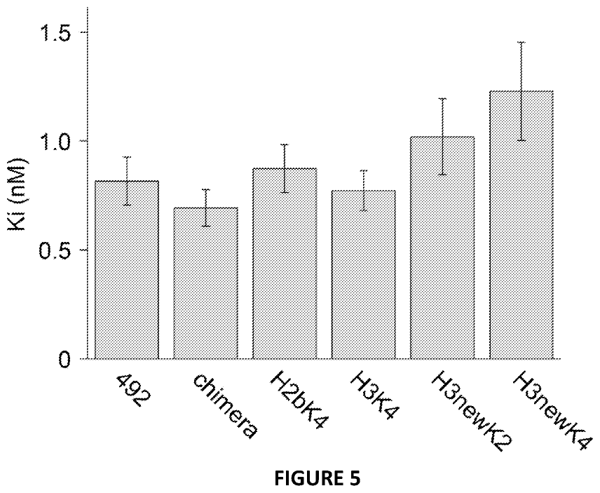

As shown in FIGS. 4A-4F and FIG. 5, the humanized antibodies tested, H3newK2 (FIG. 4B), H3newK4 (FIG. 4C), H3K4 (FIG. 4E) and H2bK4 (FIG. 4F) have sub-nanomolar Ki values, similar to the original murine antibody 492 (FIG. 4A).

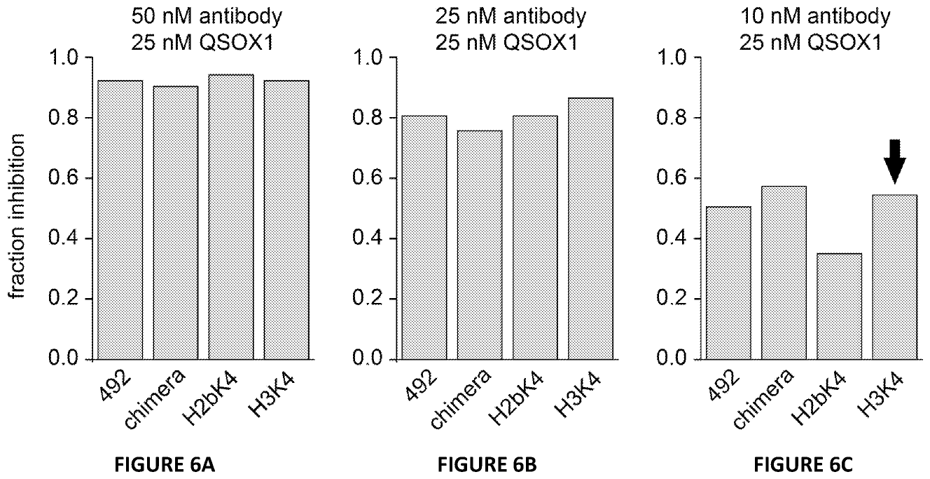

In another assay measuring QSOX1 activity on DTT, 50 μL reactions were set up in 96-well plates with the following final concentrations of components: 25 nM QSOX1 and 10, 25 or 50 nM antibody. The reaction was initiated by addition of DTT to a final concentration of 300 μM. Reactions were stopped after 20 min at room temperature by adding 1 mM of 5,5-dithiobis-(2-nitrobenzoic acid) (DTNB), which reacts with residual DTT and produces the chromogenic product 5-thio-2-nitrobenzoate (TNB) with absorption at 412 nm. Parallel control reactions were conducted without QSOX1 or without antibody, and fraction inhibition was calculated using the dynamic range established by these control measurements. The results, shown in FIGS. 6A-6C, clearly demonstrate that two of the designed antibodies, H2bK4 and H3K4, showed equivalent levels of QSOX1 inhibition to the parental mouse antibody when added at stoichiometric ratios (FIG. 6B) and one (H3K4) exhibited substantial levels of inhibition even at sub stoichiometric ratios (FIG. 6C).

### Example 4. Differential Scanning Fluorimetry (DSF) Thermal Stability Assays of the Humanized Variants and Fab Fragments Derived from them

This assay is performed in order to assess the thermal stability of the antibodies and their fragments. Humanized antibodies or fragments thereof at a concentration of 0.5 mg/ml in PBS, are loaded into DSF capillaries in duplicate. A temperature ramp from 20 to 94 degrees Celsius is performed, and changes is tryptophan fluorescence is monitored to indicate unfolding. For Fab fragments, antibody cleavage is performed with papain, the Fc fragment is removed using protein A, and the Fab is subjected to DSF according to a similar protocol.

### Example 5. H3K4 Stability in Human Blood Plasma

The stability and functionality of humanized antibody H3K4 after long incubation in human blood plasma was tested, in comparison with the murine antibody 492 (mAb492.1). Human blood collected from 2 donors with heparin as an anticoagulant was centrifuged for 15 min at 300 g. Plasma was collected and used as a native source of QSOX1 for inhibition reactions as follows. Antibody (H3K4 or MAb492.1) was added to plasma 1:10 volume:volume to achieve a final antibody concentration of 250 nM. As controls, PBS was added instead of antibody to a plasma sample, or PBS was added instead of plasma to a sample containing Mab492.1. The mixtures were incubated at 37° C. Aliquots of 11 μl were removed at various times and added to 83 μl buffer (50 mM potassium phosphate buffer, pH 7.5, 65 mM NaCl, and 1 mM EDTA). Dithiothreitol was then added from a 5 mM stock to achieve a final concentration of 300 μM and initiate the reactions. Reactions were allowed to proceed for 30 min at room temperature. 50 μl were then removed and added to 750 μl of the above buffer containing 5,5′-dithiobis-2-nitrobenzoic acid (DTNB) at a final concentration of 63 μM. After 10 min, absorbance was measured at 412 nm. Three technical replicates were performed at each timepoint for each of two biological replicates. Error bars indicate standard deviations. Assays were performed by spiking samples after the indicated incubation times with DTT, allowing endogenous plasma QSOX1 to oxidize thiols at room temperature for half an hour, and then reacting the remaining thiols with the colorimetric reagent DTNB (absorbance maximum 412 nm).

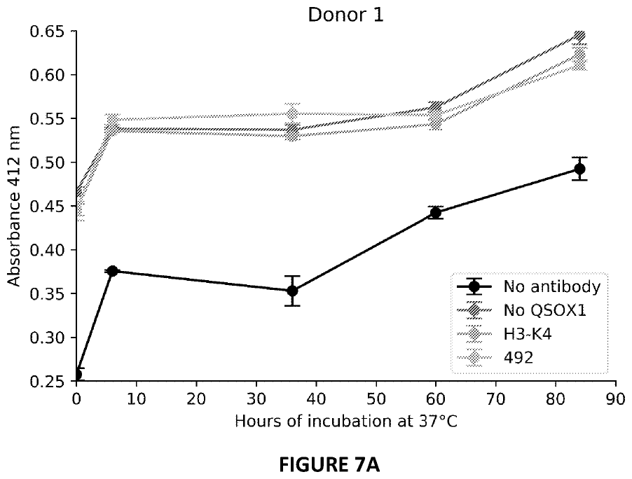

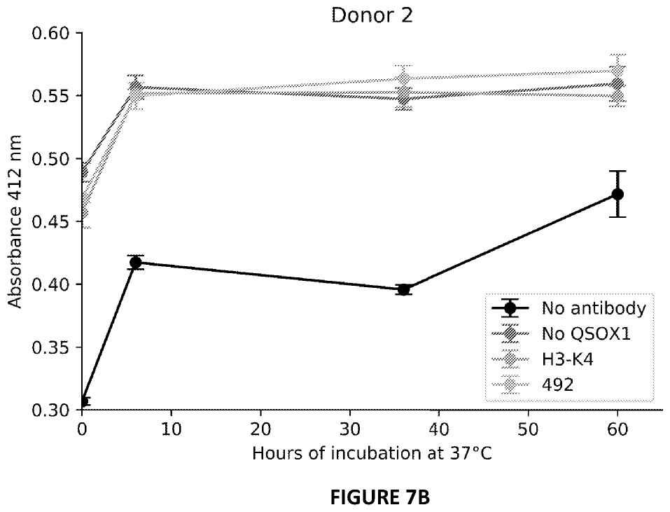

As demonstrated in FIGS. 7A and 7B, blood plasma QSOX1 was inhibited by the humanized antibody H3K4 and by the parental murine antibody MAb492.1. The dynamic range of the experiment decreased over time, apparently due to loss of plasma QSOX1 activity during incubation. Nevertheless, all remaining QSOX1 activity was effectively inhibited by the antibodies, indicating that the humanized antibody H3K4 was stable and functional for days at 37° C. in plasma, similarly to the parental murine antibody MAb492.1.

### Example 6. Inhibition of Extracellular Matrix (ECM) Formation in Fibroblast Cultures

The humanized antibodies are tested for their ability to inhibit formation of ECM in human WI-38 fibroblasts. Antibody is added at a concentration of 250 nM to sub-confluent (˜70%) culture of fibroblasts. After 4 days, cells are fixed and labeled for fibronectin using specific antibodies. Secondary fluorescent antibodies are used for visualization of fibronectin network intensity and appearance that indicate the level of ECM formation.

### Example 7. Tumor Cell Adhesion Assay to Fibroblast ECM

The adhesion of tumor cells to fibroblast ECM formed in the presence or absence of QSOX1 inhibitory humanized antibodies is tested. Humanized antibodies or fragments thereof are added at a concentration of 250 nM to sub-confluent (˜70%) WI-38 fibroblasts. After four days, medium is replaced, and fluorescently labeled human epithelial cancer cells (MDA-MB-231) are added to the fibroblast culture and allowed to adhere for 30-60 minutes. Following rigorous washing, remaining MDA-MB-231 cells are quantified by fluorescence imaging.

### Example 8. Tumor Cell Migration Assay

To assess tumor cell migration through fibroblasts and associated ECM formed in the presence or absence of QSOX1 inhibitory antibodies, human WI-38 fibroblasts are grown on Transwell inserts with pores of 8 micrometers. Humanized antibodies are added at a concentration of 250 nM when the fibroblasts are sub-confluent (˜70%). After four days, medium is exchanged and fluorescently labeled MDA-MB-231 cells are added to the upper chamber and allowed to migrate during one day toward the higher concentration of serum in the lower compartment. Transwell inserts are removed, and the cells that have migrated through the pores to the bottom of the membrane are quantified by fluorescence imaging.

### Example 9. In Vivo Testing of the Humanized Antibodies

The humanized antibodies of the present invention are tested in vivo using animal models known in the art for testing antibodies against a human protein. One animal model used for testing the humanized QSOX1 inhibitory antibodies of the present invention is the xenograft breast cancer model described in Feldman et al., (2020 ibid).

According to a non-limited in vivo protocol human breast adenocarcinoma MDA-MB-231 cells (200,000 in 200 ml 1:1 HBSS: Cultrex®) are orthotopically injected into mammary fat pads of 8-week old female nude mice. Treatments are started 3 days after cell injection, once tumors were visible. Mice are treated either with control antibody, humanized QSOX1-specific antibody, chemotherapy, or a combination of humanized QSOX1-specific antibody and chemotherapy. As this model involves human-derived tumor cells engrafted into mice, both humanized anti-QSOX1 antibody (25 mg/kg or 10 mg/kg) and anti-mouse QSOX1 (MAb316.1, 30 mg/kg) are administered to groups receiving QSOX1-specific antibodies. Tumor size is estimated externally using a caliper during the course of the experiment. Tumors are excised at experiment endpoint (typically 28 days), and tumor volumes are measured.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation.

1. A humanized antibody that specifically binds human Quiescin Sulfhydryl Oxidase 1 (QSOX1), or an antigen-binding fragment thereof, wherein the humanized antibody or the antigen-binding fragment comprises three complementarity-determining regions (CDRs) of a heavy-chain (HC) variable region wherein, -HC CDR1 comprises the sequence VSGFSLTGYGVN (SEQ ID NO: 3); HC CDR2 comprises the sequence WLGMIWGDGRTD (SEQ ID NO: 4); HC CDR3 comprises the sequence ASDYYGSGSFAY (SEQ ID NO: 5); and three CDRs of a light-chain (LC) variable region, wherein LC CDR1 comprises the sequence KASQDVSTAVA (SEQ ID NO: 6); LC CDR2 comprises the sequence LLIHSASYRY (SEQ ID NO: 7); and LC CDR3 comprises the sequence QQHYSIPLT (SEQ ID NO: 8), and wherein said antibody or antigen-binding fragment has an inhibitory constant (Ki) of 5×10⁻⁹M or lower to human QSOX1.

2-5. (canceled)

6. The humanized antibody or antigen-binding fragment according claim 1, comprising a heavy-chain variable region selected from the group consisting of: IGHV2-5-IGHJ6 (SEQ ID NO: 9); IGHV2-5-IGHJ2 (SEQ ID NO: 10); and IGHV3-64D-IGHJ1 (SEQ ID NO: 11); and a light-chain variable region selected from the group consisting of:
IGKV1-33-IGKJ1 (SEQ ID NO: 12); IGKV2D-29-IGKJ3 (SEQ ID NO: 13); IGKV3D-7-IGKJ1 (SEQ ID NO: 14); IGKV4-1-IGKJ5 (SEQ ID NO: 15); and IGKV1-13-IGKJ1 (SEQ ID NO: 16); or comprising an analog or derivative thereof having at least 90% sequence identity with any of said heavy chain or light chain variable region sequences.

7-8. (canceled)

9. The humanized antibody according to claim 1, comprising a heavy-chain having a sequence selected from the group consisting of: SEQ ID NO: 18 (H3), SEQ ID NO: 19 (H3new), and SEQ ID NO: 17 (H2b) and a light-chain having a sequence selected from the group consisting of: SEQ ID NO: 21 (k4) and SEQ ID NO: 20 (k2), an analog or derivative thereof having at least 90% sequence identity with said antibody sequence.

10. The humanized antibody according to claim 1, selected from the group consisting of:
i) a humanized antibody denoted H3K4 comprising the heavy chain sequence set forth in SEQ ID NO: 18 (H3), and the light chain sequence set forth in SEQ ID NO: 21(K4);
ii) a humanized antibody denoted H2bK4 comprising the heavy chain sequence set forth in SEQ ID NO: 17 (H2b), and the light chain sequence set forth in SEQ ID NO: 21 (K4);
iii) a humanized antibody denoted H3newK2 comprising the heavy chain sequence set forth in SEQ ID NO: 19 (H3new), and the light chain sequence set forth in SEQ ID NO: 20 (K2); and
iv) a humanized antibody denoted H3newK4 comprising the heavy chain sequence set forth in SEQ ID NO: 19 (H3new), and the light chain sequence set forth in SEQ ID NO: 21 (K4);
or an analog or derivative thereof having at least 90% sequence identity with said antibody sequence.

11. The humanized antibody according to claim 1, comprising a human IgG1 region and a human kappa light chain region.

12. (canceled)

13. An antigen-binding fragment according to claim 1, wherein the antigen-binding fragment is a Fab or a scFv.

14. A conjugate comprising at least one humanized antibody or antigen-binding fragment according to claim 1.

15-16. (canceled)

17. The humanized antibody or antigen-binding fragment according to claim 1, wherein the antibody or antigen-binding fragment is capable of inhibiting the human QSOX1 activity of oxidizing cysteine residues in ECM proteins.

18. A polynucleotide sequence encoding at least one chain of a humanized antibody or an antigen-binding fragment thereof, according to claim 1.

19. (canceled)

20. The polynucleotide sequence according to claim 18, wherein the polynucleotide sequence is selected from SEQ ID NOS: 23-29 or an analog or derivative thereof having at least 90% sequence identity with said polynucleotide sequence.

21. A construct comprising at least one polynucleotide sequence according to claim 20, or a host cell comprising said construct.

22. (canceled)

23. A pharmaceutical composition comprising as an active ingredient, at least one humanized antibody or antigen-binding fragment or conjugate according to claim 1, and at least one pharmaceutical acceptable excipient, diluent, salt, or carrier.

24-30. (canceled)

31. A method of inhibiting the activity of human QSOX1 in a subject in need thereof, comprising administering to said subject a therapeutically effective amount of a humanized antibody or an antigen-binding fragment or a conjugate according to claim 1.

32. A method of treating a laminin-associated disease or condition in a subject in need thereof, comprising administering to said subject a therapeutically effective amount of a humanized antibody or an antigen-binding fragment or a conjugate according to claim 1.

33. The method of claim 32, wherein the laminin-associated disease or condition is a tumor or a cancer.

34. The method of claim 33, wherein the disease is a solid tumor or a metastatic tumor or cancer or an advanced or metastatic fibroblast activation protein-positive cancer.

35. (canceled)

36. The method of claim 33, wherein the cancer is selected from the group consisting of a prostate cancer, a lung cancer, a breast cancer, a cervical cancer, an urachus cancer, a vaginal cancer, a colon cancer, an esophagus cancer, a pancreatic cancer, a throat cancer, a stomach cancer and a myeloid leukemia.

37. The method according to claim 33, further comprising administering or performing at least one additional anti-cancer therapy.

38-39. (canceled)

40. A method of determining or quantifying the presence of human QSOX1 in a sample, the method comprising contacting a biological sample with a humanized antibody or antigen-binding fragment or conjugate according to claim 1 and measuring the level of complex formation.

41. (canceled)

42. A kit for measuring the expression or presence of human QSOX1 in biological sample comprising at least one humanized antibody or antigen-binding fragment according to claim 1.

43-44. (canceled)
